(19) United States
(12) Patent Application Publication
(10) Pub. No.: US 2023/0360729 A1
(43) Pub. Date: Nov. 9, 2023

(54) COMPUTER-IMPLEMENTED METHOD FOR PROVIDING NUCLEIC ACID SEQUENCE DATA SET FOR DESIGN OF OLIGONUCLEOTIDE

(71) Applicant: SEEGENE, INC., Seoul (KR)

(72) Inventors: Do Hee KIM, Gyeonggi-do (KR); Hyeon Joo LEE, Seoul (KR)

(21) Appl. No.: 18/021,678

(22) PCT Filed: Aug. 31, 2021
(86) PCT No.: PCT/KR2021/011636

§ 371 Date: Feb. 16, 2023

(30) Foreign Application Priority Data
Aug. 31, 2020 (KR) 10-2020-0110636

(51) Int. Cl. G16B 25/20; G16B 5/00; G16B 20/20; G16B 30/10; G16B 50/30; C12Q 1/6876

(52) U.S. Cl. CPC G16B 25/20; G16B 5/00; G16B 20/20; G16B 30/10; G16B 50/30; C12Q 1/6876

(57) ABSTRACT

The present invention relates to a computer-implemented method for providing a nucleic acid sequence data set for the design of an oligonucleotide used to detect a target nucleic acid molecule of an organism of interest. In the present invention, nucleic acid sequence data retrieved by synonyms for a target nucleic acid molecule are sorted according to the taxonomic name and/or taxonomic identification (ID); taxonomic representative sequences are selected among nucleic acid sequence data having the same taxonomic name and/or taxonomic ID; and the selected taxonomic representative sequences are grouped according to the homology to select a group representative sequence in each group; and then nucleic acid sequence data having a homology of a predetermined value or more with the group representative sequence are provided.

### CROSS-REFERENCE TO RELATED APPLICATIONS

This application claims priority from Korean Patent Application No. 2020-0110636, filed on Aug. 31, 2020 in the Korean Intellectual Property Office, the disclosure of which is incorporated herein by reference.

### BACKGROUND OF THE INVENTION

### Field of the Invention

The present invention relates to a computer-implemented method for providing a nucleic acid sequence data set for the design of an oligonucleotide used to detect a target nucleic acid molecule of an organism of interest.

### Description of the Related Art

A 21st century health care paradigm has been changed from an era of public health and an era of disease treatment to an era of health lifespan extension through disease prevention and management. With such a global trend changing from a therapeutic medicine to a preventive medicine, a demand for in vitro diagnostics (IVD) is increasing. A global population aging and emergence of new viruses are another factor in the growth of the IVD market. Moreover, the scope of in vitro diagnosis on a patient that precedes the determination of treatment or prescription for the patient is also expanding since the treatment methods are also changed to personalized treatment.

Molecular diagnosis is the fastest growing field in the in vitro diagnostics (IVD) industry and is important in patient care continuity. Compared with other diagnosis platforms where disease portfolios overlap, molecular diagnosis has advantages of excellent test precision, miniaturization, and fast processing time. Thanks to the strength of this molecular diagnostic technology, general diagnostic items which have been carried out by chemical and immunological tests in the past are recently being gradually replaced with molecular diagnostic test items.

Typical techniques used in molecular diagnostics are polymerase chain reaction (PCR), next-generation sequencing (NGS), microarray, fluorescent in situ hybridization, and the like.

Nucleic acid amplification method, which is known as a polymerase chain reaction (hereinafter, referred to as “PCR”), involves repeated cycles of denaturation of double-stranded DNA, oligonucleotide primer annealing to the DNA template, and primer extension by a DNA polymerase (Mullis et al., U.S. Pat. Nos. 4,683,195, 4,683,202, and 4,800,159; and Saiki et al., (1985) Science 230, 1350-1354).

PCR-based techniques are widely used not only for the amplification of target DNA sequences but also for scientific applications or methods in biology and medical research. For example, detection of a target sequence, reverse transcriptase PCR (RT-PCR), differential display PCR (DD-PCR), cloning of known or unknown genes using PCR, rapid amplification of cDNA ends (RACE), arbitrary priming PCR (AP-PCR), multiplex PCR, SNP genomic typing, and PCR-based genomic analysis are available.

These molecular diagnostic techniques analyze pathogens or risk factors for disease by identifying the presence or sequence of a target nucleic acid molecule in the sample, and in most cases, the sequence of the target nucleic acid molecule is selectively amplified for analysis. In order to carry out this molecular diagnosis, it is important to design oligonucleotides used for the amplification and detection of the target nucleic acid molecule.

The oligonucleotides (probe and/or primer) used for detection of target nucleic acid molecules need to have suitable degrees of specificity and detection, be fit for a specific detection method, and comply with the conditions set by the analysts. It is therefore very important to design oligonucleotides to be suitable for analytical purposes.

Most target nucleic acid sequences of genes of higher animals, such as humans and mammals, and genes of various bacteria and viruses classified as pathogens include sequence variations among individuals. In particular, RNA viruses are known to have high sequence variability (genetic diversity). More sophisticated oligonucleotide designs are required to detect target nucleic acid molecules with high genetic diversity with an appropriate coverage.

There have been various attempts to design oligonucleotides to detect target nucleic acid molecules with genetic diversity. A common method of designing such oligonucleotides is to find out conserved regions of multiple target nucleic acid molecules with genetic diversity and to design oligonucleotides to be hybridized with these conserved regions (Wang, D et al., Proc. Natl Acad. Sci. USA, 99:15687-15692(2002)).

The designing of oligonucleotides for the conserved regions required the retrieval of target nucleic acid sequences containing many sequence variants classified as the same species. Conventional methods disclose a new attempt to process several target nucleic acid sequence data of a target nucleic acid molecule and find conserved regions using this process, but no methodological progress has been made in the retrieval of homologous sequences provided for the design of oligonucleotides and the processing thereof, and still depends on the personal knowledge and experience of the researchers retrieving sequences.

With such manual-based sequence retrieval, the specificity of the designed oligonucleotide to a target nucleic acid sequence, and the coverage of the target nucleic acid sequences detectable by the oligonucleotide may be limited by the researcher's capabilities, and the development time is increased by the retrieval of sequences.

To solve these problems, it was necessary to develop an automated new method for efficiently retrieving target nucleic acid sequence data of a target nucleic acid molecule.

FIG. 1 is a flow chart showing the process of providing a target nucleic acid sequence data set for a target nucleic acid molecule according to the method (WO2019/212238) previously filed by the present applicant. According to the flow chart of FIG. 1, nucleic acid sequence data are retrieved using keywords, such as a name of a target nucleic acid molecule; the retrieved nucleic acid sequence data are sorted according to the sequence length to determine the longest sequence as a representative sequence; nucleic acid sequence data having a homology of a predetermined degree or more with the representative sequence are grouped; and the nucleic acid sequence data retrieved by using the keywords and the nucleic acid sequence data having homology with the representative sequence are provided as a target nucleic acid sequence data set for the target nucleic acid molecule. The results of aligning of multiple target nucleic acid sequences of the target nucleic acid sequence data are shown in FIG. 2.

As can be seen from FIG. 2, as a result of providing a nucleic acid sequence data set for a target nucleic acid molecule by the conventional method, the number of representative sequences was 25, and the sequences retrieved by using the representative sequences were not properly aligned due to a difference in homology. Therefore, analysts should spend unnecessary time, for example, for reviewing aligned nucleic acid sequences.

Accordingly, in order to design an oligonucleotide used to detect a target nucleic acid molecule of an organism of interest, the present inventors recognized the need for development of a method for providing a nucleic acid sequence data set for the design of an oligonucleotide, the set being capable of retrieving multiple target nucleic acid sequences for the target nucleic acid molecule without omission and properly forming alignment results so as to use the retrieved multiple target nucleic acid sequences in the design of an oligonucleotide.

Throughout this application, various patents and publications are referenced and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have endeavored to develop a computer-implemented method capable of effectively providing a nucleic acid sequence data set for use in the design of an oligonucleotide used in the amplification or detection of a target nucleic acid molecule while overcoming the problems of the conventional method. As a result, the present inventors confirmed that the alignment results of multiple nucleic acid sequences were properly formed so as to design an oligonucleotide, by sorting nucleic acid sequence data, retrieved by synonyms for a target nucleic acid molecule, according to taxonomic name and/or taxonomic identification (ID) and selecting taxonomic representative sequences among nucleic acid sequence data having the same taxonomic name and/or taxonomic ID, grouping the selected taxonomic representative sequences according to homology and selecting a group representative sequence for each group, and providing nucleic acid sequence data having a homology of a predetermined value or more with the group representative sequence, and therefore, the present inventors completed the present invention.

Accordingly, it is an object of the present invention to provide a computer-implemented method for providing a nucleic acid sequence data set for the design of an oligonucleotide used to detect a target nucleic acid molecule of an organism of interest.

It is another object of the present invention to provide a computer-readable storage medium containing instructions to implement a process to perform a method for providing a nucleic acid sequence data set for the design of an oligonucleotide used to detect a target nucleic acid molecule of an organism of interest.

Other objects and advantages of the present invention will become apparent from the detailed description below taken in conjugation with the appended claims and drawings.

### BRIEF DESCRIPTION OF THE DRAWINGS

FIG. 1 is a flow chart showing a process of providing a target nucleic acid sequence data set according to the conventional method (WO2019/212238).

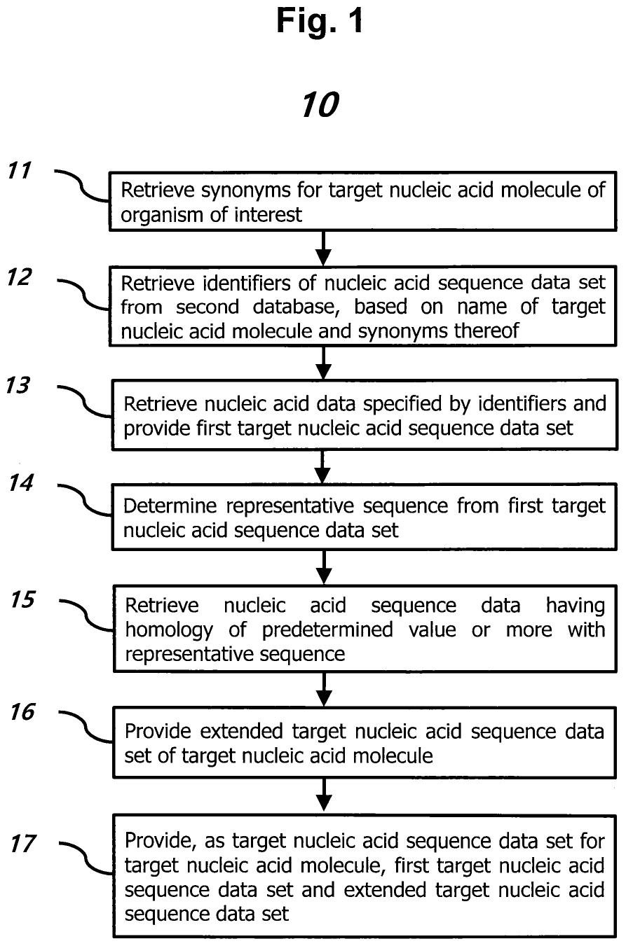

FIG. 2 shows the alignment results of a nucleic acid sequence data set for the design of an oligonucleotide used to detect sopB gene of Salmonella enterica according to the conventional method.

FIG. 3 is a flow chart showing a process of providing a nucleic acid sequence data set for the design of an oligonucleotide used to detect a target nucleic acid molecule of an organism of interest according to an embodiment of the present invention.

FIG. 4 shows that as a result of inputting the name of a target nucleic acid molecule (ompA) and the name of an organism of interest (Chlamydophila pneumoniae) in the user interface (UI), the gene information summary record was retrieved from a gene database of the National Center for Biotechnology Information (NCBI) or a gene database constructed by downloading the gene database, and the protein name as a synonym of the target nucleic acid molecule is retrieved from gene description in the record.

FIG. 5 shows that when the organism of interest is Enterobacter cloacae complex and the target nucleic acid molecule is ompX, identifiers of nucleic acid records are retrieved. Referring to FIG. 5, accession No and GI No as identifiers can be seen.

FIG. 6 is a captured picture of a part of a nucleic acid record that appears when clicking on the title for Accession: CP017990.1 in FIG. 5. In the nucleic acid record, gene, CDS, /gene, /note, /product, and the like represent a descriptor.

FIG. 7 shows a procedure of retrieving nucleic acid sequence data specified by identifiers retrieved using the name of a target nucleic acid molecule (ompX) and a synonym (outer membrane protein) of an organism of interest (Enterobacter cloacae complex), sorting nucleic acid sequence data having the same taxonomic ID according to sort criteria, and selecting a taxonomic representative sequence.

FIG. 8 shows a user interface (UI) in which sopB is input as the name of a target nucleic acid molecule of Salmonella enterica according to an embodiment of the present invention. Salmonella enterica as an organism of interest and the taxonomic number (Taxonomic ID: 28901) thereof are input by clicking on the In/Exclusivity of the UI.

FIG. 9 shows a user interface (UI) in which sopB as the name of a target nucleic acid molecule of Salmonella enterica and a protein name (inositol phosphatase) as a synonym thereof were input according to an embodiment of the present invention. Salmonella enterica as an organism of interest and the taxonomic number (Taxonomic ID: 28901) thereof are input by clicking on the In/Exclusivity of the UI.

FIG. 10 shows the alignment results of a target nucleic acid sequence data set for the target nucleic acid molecule (sopB) of the organism of interest (Salmonella enterica), provided according to an embodiment of the present invention.

FIG. 11 shows the alignment results of a target nucleic acid sequence data set for the target nucleic acid molecule (sopB) of the organism of interest (Salmonella enterica), provided according to another embodiment of the present invention.

FIG. 12 shows the alignment results provided after an analyst reviewed the alignment results of a target nucleic acid sequence data set for the target nucleic acid molecule (sopB) of the organism of interest (Salmonella enterica), provided according to the conventional method (PCT Publication No. WO2019/212238), and then ran the program four times.

### DETAILED DESCRIPTION OF THIS INVENTION

In an aspect of the present invention, there is provided a computer-implemented method for providing a nucleic acid sequence data set for the design of an oligonucleotide used to detect a target nucleic acid molecule of an organism of interest, the method including:
(a) receiving a name of the target nucleic acid molecule and a name of the organism of interest and retrieving synonyms for the target nucleic acid molecule of the organism of interest;
(b) retrieving nucleic acid sequence data included in nucleic acid records, wherein each of the nucleic acid records is associated with the organism of interest and comprises a descriptor in which at least one of the name of the target nucleic acid molecule and the retrieved synonyms are described;
(c) sorting the retrieved nucleic acid data according to taxonomic name and/or taxonomic identification (ID) and selecting taxonomic representative sequences among nucleic acid sequence data having the same taxonomic name and/or taxonomic ID;
(d) grouping the selected taxonomic representative sequences according to homology and selecting a group representative sequence for each group; and
(e) retrieving nucleic acid sequence data having a homology of a predetermined value or more with the group representative sequence to provide the retrieved nucleic acid sequence data as a nucleic acid sequence data set for the design of an oligonucleotide.

The present inventors have endeavored to develop a computer-implemented method capable of effectively providing a nucleic acid sequence data set for use in the design of an oligonucleotide used in the amplification or detection of a target nucleic acid molecule while overcoming the problems of the conventional method. As a result, the present inventors confirmed that the alignment results of multiple nucleic acid sequences were properly formed so as to design an oligonucleotide, by sorting nucleic acid sequence data, retrieved by synonyms for a target nucleic acid molecule, according to taxonomic name and/or taxonomic identification (ID) and selecting taxonomic representative sequences among nucleic acid sequence data having the same taxonomic name and/or taxonomic ID, grouping the selected taxonomic representative sequences according to homology and selecting a group representative sequence for each group, and providing nucleic acid sequence data having a homology of a predetermined value or more with the group representative sequence.

As used herein, the term “organism of interest” refers to an organism that includes a target nucleic acid molecule to be intended to amplify or detect by using an oligonucleotide (e.g., primers or probe).

As used herein, the term “organism” refers to an organism that belongs to the biological taxonomic system, for example, kingdom, division, class, order, family, genus, species, subspecies, varieties, variant, subtype, genotype, serotype, strain, isolate, or cultivar. Examples of the organism include prokaryotic cells (e.g., Mycoplasma pneumoniae, Chlamydophila pneumoniae, Legionella pneumophila, Haemophilus influenzae, Streptococcus pneumoniae, Bordetella pertussis, Bordetella parapertussis, Neisseria meningitidis; Listeria monocytogenes, Streptococcus agalactiae, Campylobacter, Clostridium difficile, Clostridium perfringens, Salmonella, Escherichia coli, Shigella, Vibrio, Yersinia enterocolitica, Aeromonas, Chlamydia trachomatis, Neisseria gonorrhoeae, Trichomonas vaginalis, Mycoplasma hominis, Mycoplasma genitalium, Ureaplasma urealyticum, Ureaplasma parvum, Mycobacterium tuberculosis), eukaryotic cells (e.g., protozoa and parasites, fungi, yeast, higher plants, lower animals, and higher animals including mammals and humans), viruses, or viroids. Examples of the parasites in the eukaryotic cells include Giardia lamblia, Entamoeba histolytica, Cryptosporidium, Blastocystis hominis, Dientamoeba fragilis, and Cyclospora cayetanensis. Examples of the viruses are influenza A virus (Flu A), influenza B virus (Flu B), respiratory syncytial virus A (RSV A), respiratory syncytial virus B (RSV B), parainfluenza virus 1 (PIV 1), parainfluenza virus 2 (PIV 2), parainfluenza virus 3 (PIV 3), parainfluenza virus 4 (PIV 4), metapneumovirus (MPV), human enterovirus (HEV), human bocavirus (HBoV), human rhinovirus (HRV), coronavirus, and adenovirus, which cause respiratory diseases; and noroviruses, rotaviruses, adenoviruses, astroviruses, and sapoviruses that cause gastrointestinal diseases. Examples of the viruses include human papillomavirus (HPV), middle east respiratory syndrome-related coronavirus (MERS-CoV), dengue virus, herpes simplex virus (HSV), human herpes virus (HHV), Epstein-Barr virus (EMV), varicella zoster virus (VZV), cytomegalovirus (CMV), HIV, hepatitis virus, and poliovirus.

As used herein, the term “target nucleic acid molecule”, “target molecule”, or “target nucleic acid” refers to a nucleotide molecule in an organism to be detected. A target nucleic acid molecule is generally given a particular name, and includes the whole genome and all nucleotide molecules constituting the genome (e.g., genes, pseudogenes, non-coding sequence molecules, untranslated region, and some regions of the genome). The target nucleic acid molecule includes, for example, nucleic acids of an organism.

The target nucleic acid molecule herein may mean the entirety or a part of a nucleic acid molecule to be detected. The target nucleic acid molecule herein may mean one functional unit of the nucleic acid molecule. The functional unit may be a gene. The gene is a physical or functional unit of genetic information consisting of DNA or RNA. The gene encompasses both regions encoding proteins and regions that do not encode proteins. As used herein, the term “target gene” may be used interchangeably with “target nucleic acid molecule” when the target nucleic acid molecule means a gene part as a functional unit in a physical nucleic acid molecule.

As used herein, the term “detection” refers to the measurement that provides a qualitative or quantitative indication of the presence or absence of a target nucleic acid molecule. The detection includes identification, determination, or analysis.

As used herein, the term “oligonucleotide” refers to a linear oligomer of native or modified monomers or linkages. The oligonucleotide includes deoxyribonucleotides and ribonucleotides, can specifically hybridize with a target nucleotide sequence, and is naturally present or artificially synthesized. The oligonucleotide is especially a single chain for maximal efficiency in hybridization. Specifically, the oligonucleotide is an oligodeoxyribonucleotide. The oligonucleotide used in the present invention may include naturally occurring dNMPs (i.e., dAMP, dGMP, dCMP, and dTMP), nucleotide analogs, or derivatives. The oligonucleotide may also include ribonucleotides. Examples of the oligonucleotide of the present invention may include backbone-modified nucleotides, such as peptide nucleic acid (PNA) (M. Egholm et al., Nature, 365:566-568 (1993)), locked nucleic acid (LNA) (WO1999/014226), bridged nucleic acid (BNA) (WO2005/021570), phosphorothioate DNA, phosphorodithioate DNA, phosphoramidate DNA, amide-linked DNA, MMI-linked DNA, 2′-O-methyl RNA, alpha-DNA and methylphosphonate DNA, sugar-modified nucleotides, such as 2′-O-methyl RNA, 2′-fluoro RNA, 2′-amino RNA, 2′-O-alkyl DNA, 2′-O-allyl DNA, 2′-O-alkynyl DNA, hexose DNA, pyranosyl RNA, and anhydrohexitol DNA, and base-modified nucleotides, such as C-5 substituted pyrimidine (the substituent including fluoro-, bromo-, chloro-, iodo-, methyl-, ethyl-, vinyl-, formyl-, ethynyl-, propynyl-, alkynyl-, thiazolyl-, imidazolyl-, and pyridyl-), 7-deazapurines with C-7 substituent (substituent including fluoro-, bromo-, chloro-, iodo-, methyl-, ethyl-, vinyl-, formyl-, alkynyl-, alkenyl-, thiazolyl-, imidazolyl-, and pyridyl-), inosine, and diaminopurine. Especially, the term “oligonucleotide” used herein is a single strand composed of a deoxyribonucleotide. The term “oligonucleotide” includes oligonucleotides that hybridize with cleavage fragments occurring depending on a target nucleic acid sequence.

According to an embodiment of the present invention, the oligonucleotide is a primer and/or a probe.

As used herein, the term “primer” refers to an oligonucleotide that can act as a point of initiation of synthesis when placed under conditions in which synthesis of primer extension products complementary to a target nucleic acid strand (a template) is induced, i.e., in the presence of nucleotides and a polymerase, such as DNA polymerase, and appropriate temperature and pH conditions. The primer needs to be long enough to prime the synthesis of extension products in the presence of a polymerase. An appropriate length of the primer is determined according to multiple factors, including temperatures, fields of application, and primer sources.

As used herein, the term “probe” refers to a single-stranded nucleic acid molecule containing a portion or portions that are complementary to a target nucleic acid sequence. The probe may also contain a label capable of generating a signal for target detection.

The oligonucleotide may have typical primer and probe structures each composed of a sequence hybridizing with a target nucleic acid sequence. Alternatively, the oligonucleotide may have distinctive structures through structural modification thereof. For example, the oligonucleotide may have a structure of Scorpion primer, Molecular beacon probe, Sunrise primer, HyBeacon probe, tagging probe, DPO primer or probe (WO 2006/095981), and PTO probe (WO 2012/096523).

The oligonucleotide may be a modified oligonucleotide, such as a degenerate base-containing oligonucleotide and/or a universal base-containing oligonucleotide wherein degenerate bases and/or universal bases are introduced into a conventional primer or probe. As used herein, the terms “conventional primer”, “conventional probe”, and “conventional oligonucleotide” refer to a typical primer, a probe, and an oligonucleotide, into which a degenerate base or non-natural base is not introduced. According to an embodiment, the degenerate base-containing oligonucleotide or universal base-containing oligonucleotide is an oligonucleotide of which at least 50%, at least 60%, at least 70%, at least 80%, at least 90% or at least 95% is not modified. According to an embodiment of the present invention, the number of degenerate bases or universal bases introduced into the conventional oligonucleotide is in the range of specifically 7 or less, 5 or less, 4 or less, 3 or less, or 2 or less. Alternatively, the use proportion of the degenerate bases and/or universal bases introduced into the conventional oligonucleotide is specifically 25% or less, 20% or less, 18% or less, 16% or less, 14% or less, 12% or less, 10% or less, 8% or less, or 6% or less. The use proportion of the degenerate bases or universal bases represents a proportion of the degenerate bases or universal bases over a total of the nucleotides of the oligonucleotide into which the degenerate bases or universal bases are introduced. The degenerate bases include a variety of degenerate bases known in the art as follows: R: A or G; Y: C or T; S: G or C; W: A or T; K: G or T; M: A or C; B: C, G or T; D: A, G or T; H: A, C or T; V: A, C or G; N: A, C, G or T. The universal bases include a variety of universal bases known in the art as follows: deoxyinosine, inosine, 7-deaza-2′-deoxyinosine, 2-aza-2′-deoxyinosine, 2′-OMe inosine, 2′-F inosine, deoxy 3-nitropyrrole, 3-nitropyrrole, 2′-OMe 3-nitropyrrole, 2′-F 3-nitropyrrole, 1-(2′-deoxy-beta-D-ribofuranosyl)-3-nitropyrrole, deoxy 5-nitropyrrole, 5-nitroindole, 2′-OMe 5-nitroindole, 2′-F 5-nitroindole, deoxy 4-nitrobenzimidazole, 4-nitrobenzimidazole, deoxy 4-aminobenzimidazole, 4-aminobenzimidazole, deoxy nebularine, 2′-F nebularine, 2′-F 4-nitrobenzimidazole, PNA-5-introindole, PNA-nebularine, PNA-inosine, PNA-4-nitrobenzimidazole, PNA-3-nitropyrrole, morpholino-5-nitroindole, morpholino-nebularine, morpholino-inosine, morpholino-4-nitrobenzimidazole, morpholino-3-nitropyrrole, phosphoramidate-5-nitroindole, phosphoramidate-nebularine, phosphoramidate-inosine, phosphoramidate-4-nitrobenzimidazole, phosphoramidate-3-nitropyrrole, 2′-O-methoxyethyl inosine, 2′-O-methoxyethyl nebularine, 2′-O-methoxyethyl 5-nitroindole, 2′-O-methoxyethyl 4-nitro-benzimidazole, 2′-O-methoxyethyl 3-nitropyrrole, and a combination thereof. More specifically, the universal base is deoxyinosine, inosine, or a combination thereof.

In order to design an oligonucleotide used to detect a particular target nucleic acid molecule, various methods known in the art may be performed. For example, multiple target nucleic acid sequences for a particular target nucleic acid molecule are retrieved and aligned, and then an oligonucleotide may be designed from each of the multiple target nucleic acid sequences according to design conditions. It is therefore important to retrieve multiple target nucleic acid sequences for a target nucleic acid molecule of an organism of interest in order to design an oligonucleotide.

An oligonucleotide to be designed includes a probe that is designed to satisfy at least one of the following conditions: (i) a Tm value of 50-85° C.; (ii) a length of 15-50 nucleotides; (iii) the exclusion of a mononucleotide (G)n run sequence in which n is at least 3; (iv) G or C at the 5′-end; and (v) a GC content of 40% or more at the 5′-end portion.

The probe design conditions include, specifically, at least two, more specifically, at least three, still more specifically, at least four, and still more specifically, five among the above-described conditions.

The Tm value among the design conditions is, for example, 50-80° C., 50-75° C., 55-80° C., 55-75° C., 60-80° C., 60-75° C., 65-80° C., or 65-75° C. Specifically, the Tm value among the design conditions is, for example, 55-80° C., 60-78° C., 63-78° C., 65-75° C., 67-75° C., or 65-73° C.

The length among the design conditions is, for example, 10-60 nucleotides, 10-50 nucleotides, 10-45 nucleotides, 10-40 nucleotides, 10-35 nucleotides, 15-60 nucleotides, 15-50 nucleotides, 15-45 nucleotides, 15-40 nucleotides, or 15-35 nucleotides.

There is the exclusion of a mononucleotide (G)ₙ run sequence in which n is, for example, at least 3 or 4.

The GC content at the 5′-end of the probe is 40% or more, specifically, 40-70%, or 40-60%. The 5′-end portion means a region within 10 nucleotides from the 5′-end of the probe.

An oligonucleotide to be designed includes a primer that is designed to satisfy at least one of the following conditions: (i) a Tm value of 40-70° C.; (ii) a length of 15-60 nucleotides; and (iii) the exclusion of a mononucleotide (G)ₙ run sequences in which n is at least 3.

The Tm value among the design conditions is, for example, 40-70° C., 50-70° C., 55-70° C., 45-65° C., 50-65° C., 55-65° C., 45-60° C., or 50-65° C. Specifically, the Tm value among the design conditions is, for example, 40-70° C., 45-65° C., 50-65° C., 50-60° C., 55-65° C., or 55-60° C.

The length among the design conditions is, for example, 15-60 nucleotides, 15-50 nucleotides, 15-45 nucleotides, 15-40 nucleotides, 15-35 nucleotides, 15-30 nucleotides, 15-25 nucleotides, 18-45 nucleotides, 18-40 nucleotides, 18-35 nucleotides, 18-30 nucleotides, or 18-25 nucleotides. Specifically, the length among the design conditions is, for example, 15-40 nucleotides, 16-40 nucleotides, 17-40 nucleotides, 18-40 nucleotides, 15-35 nucleotides, 16-35 nucleotides, 17-35 nucleotides, 18-35 nucleotides, 15-30 nucleotides, 16-30 nucleotides, 17-30 nucleotides, 18-30 nucleotides, 18-25 nucleotides, or 17-25 nucleotides.

The mononucleotide (G)n run sequence in the design conditions has a criterion, a mononucleotide (G)ₙ run sequence in which n is, for example, at least 3 or 4 being excluded.

In cases where the primer is a DPO primer developed by the present applicant (see U.S. Pat. No. 8,092,997), the description for the Tm and the length of the DPO primer disclosed in the patent document may be presented as the design conditions.

The primer design conditions include, more specifically, at least two, and still more specifically, at least three of the above-described conditions.

As used herein, the term “sequence” refers to a distinctive arrangement order of monomers within a macromolecule. As used herein, the term “nucleic acid sequence” refers to a particular target nucleic acid sequence of a nucleic acid molecule, expressed by the arrangement order of nucleotides in the nucleic acid molecule.

As used herein, the term “nucleic acid sequence” or “nucleic acid sequence data” refers to the arrangement order of nucleotides in the nucleic acid molecule or information on the arrangement order of nucleotides in the nucleic acid molecule, and may be used interchangeably. The term “nucleic acid sequence data set” refers to a set of the nucleic acid sequence data, wherein the nucleic acid sequence data set may be provided in the form of a list or alignment file of nucleic acid sequence data.

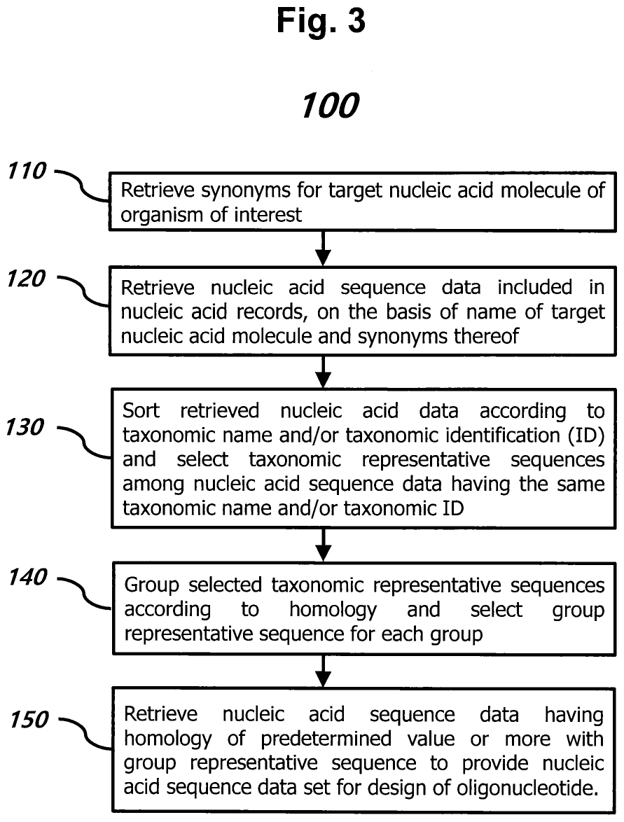

FIG. 3 is a flow chart showing the steps of performing the method according to an embodiment of the present invention. The method of the present invention will be described in detail with reference to FIG. 3 as follows.

### Step (a): Retrieving Synonyms for Target Nucleic Acid Molecule of Organism of Interest (110)

First, the method of the present invention includes step (a) of receiving a name of a target nucleic acid molecule and a name of an organism of interest and retrieving synonyms for the target nucleic acid molecule of the organism of interest.

As used herein, the term “name of target nucleic acid molecule” refers to a word or a symbol representing a target nucleic acid molecule. In the present invention, the name of the target nucleic acid molecule may be a name of a nucleotide molecule (e.g., gene, pseudo gene, non-coding sequence molecule, untranslated region, and some regions of genome). The name encompasses an official full name and a common name. The common name refers to a name that is used to represent a target nucleic acid molecule in the art to which the present invention pertains, besides the official full name. The symbol herein is a mark, a sign, a letter, or a combination of letters, which represents a target nucleic acid molecule. The symbol encompasses an official symbol and an alias. The alias refers to a nonofficial symbol that is used to identify a target nucleic acid molecule in the technical field to which the present invention pertains, besides the official symbol.

The name of the organism of interest herein refers to a scientific name or a taxonomic name of the organism according to the biological taxonomic system. The name of the organism of interest also encompasses taxonomic ID assigned to the scientific name or taxonomic name of the organism.

When the computer-implemented method of the present invention is to be performed, a user needs to input the name of the target nucleic acid molecule and the name of the organism of interest through a user interface (UI), and a computer to implement the method of the present invention through such an input receives the name of the target nucleic acid molecule and the name of the organism of interest through the user interface (UI).

Therefore, regarding the input of the name of the target nucleic acid molecule and the name of the organism of interest herein, the receiving of the name of the nucleic acid molecule and the name of the organism of interest means receiving (or inputting to a computer) a name (word or symbol) representing the target nucleic acid molecule and a name of the organism containing the target nucleic acid molecule. Through such a receipt, it is determined whether multiple target nucleic acid sequence data for which nucleic acid molecule are provided by the present method.

As used herein, the term “target nucleic acid sequence” or “target sequence” refers to a particular target nucleic acid sequence representing a target nucleic acid molecule.

One target nucleic acid molecule, for example, one target gene, may have a particular target nucleic acid sequence; otherwise a target nucleic acid molecule exhibiting genetic diversity or genetic variability may have multiple target nucleic acid sequences with diversity. The plurality of target nucleic acid sequences in the present invention are target nucleic acid sequences with sequence similarity.

The method of receiving the name of the target nucleic acid molecule and the name of the organism containing the target nucleic acid molecule is not particularly limited, and for example, the names may be received by a direct input by a user through an input device (e.g., UI), or may be received through various data storage media. Alternatively, the name of the target nucleic acid molecule and the name of the organism containing the target nucleic acid molecule may be received through wired/wireless data transmission.

On the basis of the received name of the target nucleic acid molecule and name of the organism of interest, synonyms for the target nucleic acid molecule of the organism of interest are retrieved.

The method of the present invention is for retrieving and arranging as various target nucleic acid sequence data as possible and thus allowing an oligonucleotide designed therefrom to have a broad coverage for the target nucleic acid molecule. Therefore, first, it is necessary to retrieve as many target nucleic acid sequence data as possible, and to do this, synonyms for the target nucleic acid molecule, which are to be used to retrieve target nucleic acid sequence data for the design of an oligonucleotide, are retrieved.

The synonyms for target nucleic acid molecule refers to a group of words that have the same meaning as a name or symbol that identifies or indicates a target nucleic acid molecule. The synonyms for target nucleic acid molecule refers to a group of words that include both names and symbols that may identify a target nucleic acid molecule and may encompass all of an official full name, a common name, an official symbol, and an alias.

According to an embodiment of the present invention, the synonyms for the target nucleic acid molecule are retrieved from a first database.

The synonyms for the target nucleic acid molecule may be retrieved from a database. The database refers to a collection of organized data. The database may be a collection of organized data that may be stored and accessed through the computer system. Herein, the database from which the synonyms are retrieved is called the first database for distinguishment from the other databases.

More specifically, the first database may be a gene database.

The gene database refers to a database that retrieves, classifies, and stores information about genes contained in organisms. The gene database may include names, symbols, and organism names of genes, and may include descriptions of the genes, information about nucleic acid sequences of the genes (e.g., nucleic acid sequence identifiers) and information about proteins encoded by the genes (e.g., protein names and protein identifiers). The gene database may be named a “database for providing genetic information of an organism”, and the terms “gene database” and “database for providing genetic information of an organism” may be used interchangeably with each other.

According to an embodiment, the first database may be a database providing genetic information of organisms, including titles of nucleic acid molecules, names of nucleic acid molecules, descriptions of nucleic acid molecules, names of organisms, and names of proteins encoded by nucleic acid molecules. The title is information described as a title of a record when the first database provides a user with information about one nucleic acid molecule through the record.

The first database may be a directly constructed database or a user-restricted private gene database. Alternatively, the first database may be made public. The first public database includes not only those run by national or public agencies, but also those constructed by corporations, educational institutions, and research institutes. According to an embodiment of the present invention, the first database may be a publicly accessible gene database selected from the group consisting of GenBank, EMBL, and DNA DataBank of Japan (DDBJ), or may be a gene database constructed by downloading the publicly accessible gene database.

According to an embodiment of the present invention, the first database may be a publicly accessible database that includes names of target nucleic acid molecules and organism information, or a gene database constructed by downloading the same.

According to the present invention, the synonyms for the target nucleic acid molecule are retrieved, specifically, automatically retrieved from the first database.

For example, a registerer who registers a sequence of a nucleic acid molecule in the nucleotide database enters (inputs or writes) an official full name or official symbol into a field into which a gene name of a nucleic acid molecule is to be entered (inputted or written). However, in some cases, the official full name of the nucleic acid molecule may be entered into another field, and another synonym for the nucleic acid molecule may be entered into a field into which a gene name is to be entered. Alternatively, the name of the target nucleic acid molecule, received to perform the method of the present invention, may not be the actual official full name or the official symbol of the target nucleic acid molecule but one of the other synonyms thereof. It is therefore necessary to secure as many synonyms as possible for use in search in order to secure nucleic acid sequence data for a nucleic acid molecule.

Hence, (i) as many gene information summary records as possible associated with the name of the organism of interest and the name of the target nucleic acid molecule that have been input need to be secured, and (ii) the synonyms need to be secured from the secured gene information summary records.

First, in order to secure sufficient gene information summary records, among several input fields of gene information summary records associated with the nucleic acid molecule, higher-ranked fields with a high frequency of inputs of the official full name or the official symbol of the nucleic acid molecule were analyzed. As a result, in the gene database, the fields in which the gene name of the nucleic acid molecule is described and the fields in which the name of a protein produced by the expression of the nucleic acid molecule is described were confirmed as fields with a high frequency of inputs of the official full name or official symbol of the nucleic acid molecule. Specifically, it was confirmed that one of the most effective scheme for retrieving gene information summary records of the target nucleic acid molecule is to limit the name of an organism to the organism of interest, search the name of the target nucleic acid molecule by using the name, title, or protein name of a gene as a search field, and retrieve the search result through gene information summary records.

Second, in order to secure synonyms from the acquired gene information summary records, among the input fields for the gene information summary records, fields with a high frequency of inputs of another name or symbol except for the official full name or the official symbol of the nucleic acid molecule were analyzed. As a result, in the gene information summary records, the fields in which the gene name of the nucleic acid molecule is described and the description fields in which the name of the protein is described were searched as fields with a high frequency of inputs of another name or symbol except for the official full name or official symbol of the nucleic acid molecule.

Therefore, it was confirmed that one of the most effective schemes is to retrieve information described in the gene name and gene description of the retrieved gene information summary records.

According to an embodiment, step (a) may include: (a-1) of retrieving gene information summary records, wherein each of the gene information summary records is associated with the organism of interest and includes a title, gene, or protein field in which the received name of the target nucleic acid molecule is described; and (a-2) of retrieving synonyms for the target nucleic acid molecule by retrieving information described in the name, symbol, and description of the gene information summary records.

The gene information summary record is a compilation unit of information about a particular gene. According to an embodiment of the present invention, the gene information summary record is a compilation unit of information about a particular gene, including a gene name, a protein name, and description information of a gene. The gene information summary record is also called “gene information report” or “gene report”, and the terms “gene information summary record” “gene information report” and “gene report” may be used interchangeably herein.

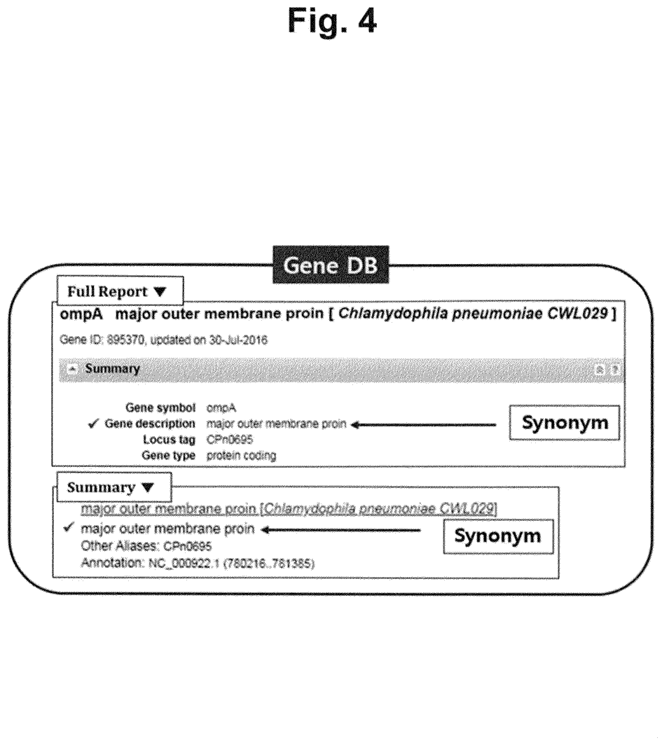

FIG. 4 shows that as a result of inputting the name of a target nucleic acid molecule (ompA) and the name of an organism of interest (Chlamydophila pneumoniae) in the UI, the gene information summary record is retrieved from the gene database of the National Center for Biotechnology Information (NCBI) or a gene database constructed by downloading the gene database and the protein name as a synonym for the target nucleic acid molecule is retrieved from gene description in the record.

According to an embodiment of the present invention, in step (a), on the basis of the specified name of the target nucleic acid molecule and name of the organism of interest, a processor of the computer may communicate with the first database via a wired or wireless network to thereby retrieve synonyms.

The receiving may be performed by receiving the name of the target nucleic acid molecule and information on the organism of interest by the user's direct input or being inputted them in the form of a file.

According to an embodiment of the present invention, step (a) may include the following steps: transmitting, by a processor, an instruction to a first database, the instruction causing the first database to transmit to a memory of the computer, a gene information summary record among gene records included in the first database, and receiving, by the processor, the gene information summary record sent from the first database in response to the instruction, wherein the gene information summary record is a record which is associated with the organism of interest and in which at least one of title, gene, and protein fields is identical to the received name of the target nucleic acid molecule; and retrieving, by the processor, the information described in the name, symbol, and description fields of the received gene information summary record, and storing the information as a synonym in the memory.

The transmitting and receiving may be performed via a wired or wireless network.

According to an embodiment of the present invention, the retrieving of synonyms in step (a) may be performed by transmitting, by a processor, an instruction to the first database, the instruction causing the first database to transmit, to a memory of the computer, information described in name, symbol, and description fields of a gene information summary record among gene records included in the first database, and retrieving, by the processor, the information sent from the first database in response to the instruction, to thereby store a synonym in the memory, wherein the gene information summary record is a record which is associated with the received organism of interest and in which at least one of the title, gene, and protein fields is identical to the received name of the target nucleic acid molecule. The synonyms stored in the memory may be stored in the form of an electronic file in a storage medium.

According to an embodiment, in step (a), the name of the target nucleic acid molecule, a name of a protein encoded by the target nucleic acid molecule, and a name of a source organism are received and the synonyms for the target nucleic acid molecule and the protein of the organism are retrieved. According to the present embodiment, in addition to the name of the target nucleic acid molecule and the name of the organism, a name of a protein encoded by the target nucleic acid molecule, known to a user, may be entered.

According to the present embodiment, the retrieving of synonyms in step (a) may be performed by transmitting, by a processor, an instruction to the first database, the instruction causing the first database to transmit, to a memory of the computer, information described in name, symbol, and description fields of a gene information summary record among gene records included in the first database, and retrieving, by the processor, the information sent from the first database in response to the instruction, to thereby store a synonym in the memory, wherein the gene information summary record is a record which is associated with the received organism of interest and in which at least one of the title, gene, and protein fields is identical to the received name of the target nucleic acid molecule.

The user may review the synonyms of the name of the target nucleic acid molecule and/or the name of the protein retrieved in such a manner and delete the synonyms determined to be inappropriate as synonyms so as not to be considered in the step to be described later.

### Step (b): Retrieving Nucleic Acid Sequence Data Included in Nucleic Acid Records (120)

The method of the present invention comprises step (b) of retrieving nucleic acid sequence data included in nucleic acid records, wherein each of the nucleic acid records is associated with the organism of interest and comprises a descriptor in which at least one of the name of the target nucleic acid molecule and the retrieved synonyms are described.

According to the present invention, the nucleic acid sequence data retrieved in step (b) are only used to select taxonomic representative sequences and group representative sequences as described below, but are not provided as a target nucleic acid sequence data set for the target nucleic acid molecule, the set being used to design an oligonucleotide.

According to an embodiment of the present invention, the nucleic acid sequence data in step (b) include nucleic acid sequence data corresponding to a part or the entirety of the target nucleic acid molecule or variant nucleic acid sequence data for the target nucleic acid molecule.

The variant nucleic acid sequence data for the target nucleic acid molecule indicate nucleic acid sequence data that contain a nucleotide sequence in which at least one nucleotide is substituted, deleted and/or added compared with the target nucleic acid sequence for the target nucleic acid molecule.

According to an embodiment of the present invention, the nucleic acid sequence data included in the nucleic acid records are retrieved from a second database.

The second database, which is distinguished from the foregoing first database, refers to a nucleotide database in which nucleic acid sequence data of several nucleic acid molecules are retrieved, classified, and stored, and the second database may be used interchangeably with the term “nucleotide database” “nucleic acid sequence database”, or “nucleic acid information collection” herein.

The second database contains nucleic acid records. The nucleic acid record may contain nucleic acid sequence data associated with a nucleic acid molecule and metadata of the nucleic acid sequence data, as a descriptor. The metadata of the nucleic acid sequence data, as a descriptor, refers to bibliographic information about the nucleic acid sequence data, and the metadata may include, for example, an identifier for nucleic acid sequence data, information about an organism including the corresponding nucleic acid molecule, keywords, information about references, such as publications in which the nucleic acid sequence data are disclosed.

The nucleic acid record may be named “nucleic acid report” or “nucleic acid information report”, and herein, the terms “nucleic acid record”, “nucleic acid report”, or “nucleic acid information report” may be used interchangeably.

As used herein, the descriptor refers to a field in which particular nucleic acid sequence data are described or identified. The descriptor may be metadata of specific nucleic acid sequence data, and specifically, the descriptor may be all fields of nucleic acid records containing a particular nucleic acid sequence data. More specifically, the descriptor may include name, definition, keywords, source organism, and reference-title.

The second database may be a directly constructed database or a user-restricted private nucleotide database. Alternatively, the second database may be made public. The second public database includes not only those run by national or public agencies, but also those constructed by corporations, educational institutions, and research institutes. According to an embodiment of the present invention, the second database may be a publicly accessible nucleotide database selected from the group consisting of GenBank, EMBL, and DDBJ, or may be a nucleotide database constructed by downloading the publicly accessible nucleotide database. According to another embodiment, the second database may be a nucleotide database including NCBI GenBank (including STS, EST, GSS, SNP, TSA, PAT, WGS and non-WGS databases), RefSeq, DDBJ, and EMBL databases, or a nucleotide database constructed by downloading the nucleotide database.

The first database and the second database of the present invention may use databases from the same organization or may use databases provided from different organizations.

According to an embodiment of the present invention, the retrieving of nucleic acid sequence data in the step (b) is performed by the method comprising the following steps:
(b-1) retrieving identifiers of nucleic acid records, wherein each of the nucleic acid records is associated with the organism of interest and comprises a descriptor in which at least one of the name of the target nucleic acid molecule and the retrieved synonyms are described; and
(b-2) retrieving nucleic acid sequence data specified by the identifiers.

Step (b-1): Retrieving Identifiers of Nucleic Acid Records

According to an embodiment of the present invention, the identifiers of the nucleic acid records are retrieved from a second database.

The identifier of the present invention refers to data used to identify specific nucleic acid sequence data. The data used as the identifier is not limited to any particular format in terms of letters, numbers, or any combination thereof. For the same nucleic acid sequence data, different identifiers may be assigned between databases. Examples of the identifier may be accession number, accession version, or GI number.

According to an embodiment of the present invention, the second database may be configured to provide a nucleic acid record containing nucleic acid sequence data and an identifier and descriptor about the nucleic acid sequence data.

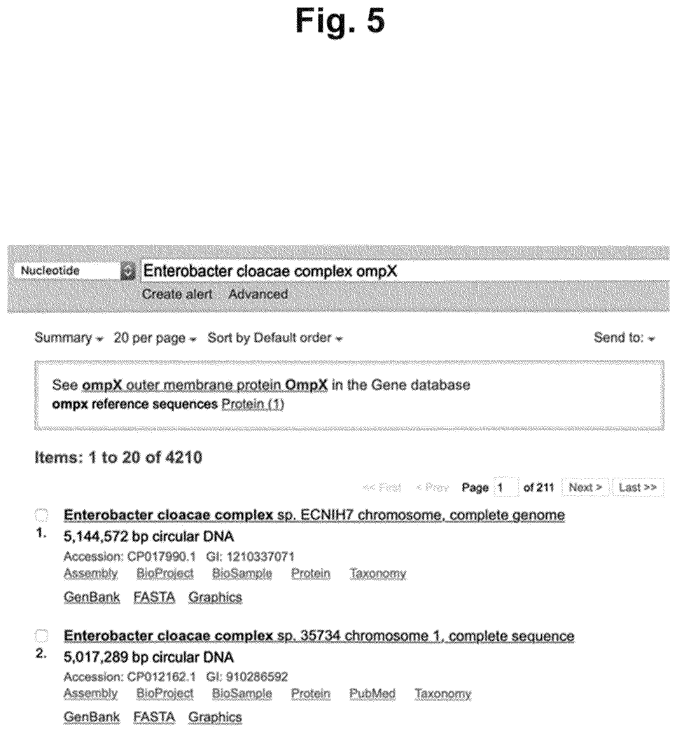

FIG. 5 shows that when the organism of interest is Enterobacter cloacae complex and the target nucleic acid molecule is ompX, identifiers of nucleic acid records are retrieved from the second database. Referring to FIG. 5, Accession No and GI No as an identifier can be shown.

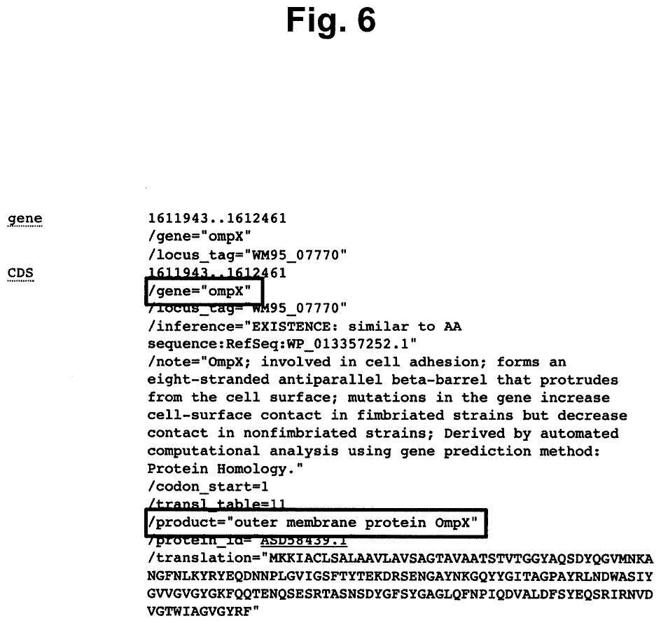

FIG. 6 is a captured picture of a part of the nucleic acid record appearing when the title of Accession: CP017990.1 in FIG. 5 is clicked. In the nucleic acid record, gene, CDS, /gene, /note, /product, or the like represents a descriptor.

According to an embodiment of the present invention, in step (b-1), a processor of the computer may communicate with the second database via a wired or wireless network, on the basis of the retrieved name of the target nucleic acid molecule and synonyms thereof and the received information about the organism, to thereby retrieve, from the second database, identifiers of nucleic acid records in which at least one of the name of the target nucleic acid molecule and the retrieved synonyms are described in a descriptor.

According to an embodiment of the present invention, in step (b-1), a processor transmits an instruction to the second database, the instruction causing the second database to transmit, to a memory of the computer, an identifier of a nucleic acid record satisfying the following conditions, and receive the information sent from the second database in response to the instruction, via a wired or wireless network, to store the information in the memory: (i) a record associated with the received organism of interest; and (ii) a nucleic acid record in which at least one of the received name of the target nucleic acid molecule or the retrieved synonyms is described in a descriptor (i.e., metadata).

The identifiers retrieved in the memory may be stored in the form of an electronic file in a storage medium.

According to an embodiment of the present invention, in step (b-1), the identifiers of the nucleic acid records are retrieved, wherein each of the nucleic acid records is associated with the organism of interest and includes a descriptor in which at least one of the name of the target nucleic acid molecule, a name of a protein, and the retrieved synonyms are described.

Step (b-2): Retrieving Nucleic Acid Sequence Data Specified by the Identifiers

The identifiers are identifiers retrieved in step (b-1), and are identifiers indicating target nucleic acid sequence data for the target nucleic acid molecule. Therefore, the nucleic acid sequence data specified by the identifiers in step (b-2) are target nucleic acid sequence data for the target nucleic acid molecule.

PCT Publication No. WO2019/212238 by the present applicant discloses that the nucleic acid sequence data specified by the identifiers are retrieved, and provided as a target nucleic acid sequence data set for the target nucleic acid molecule, and this set is used to design an oligonucleotide. However, according to the present invention, the nucleic acid sequence data retrieved in step (b-2) are only used to select taxonomic representative sequences and group representative sequences as described below, but are not provided as a target nucleic acid sequence data set for the target nucleic acid molecule, the set being used to design an oligonucleotide.

The retrieved nucleic acid sequence data may be the entirety or a part of the nucleic acid sequence data specified by the identifiers.

The nucleic acid sequence data specified by the identifiers are retrieved from the second database by using the identifiers.

As for the retrieving of the nucleic acid sequence data, nucleic acid sequences specified by the identifiers may be retrieved per se from the second database, or nucleic acid sequences may be extracted from nucleic acid records specified by the identifiers after the nucleic acid records are retrieved from the second database.

According to an embodiment of the present invention, in step (b-2), the processor transmits an instruction to the second database, the instruction requesting nucleic acid sequence data specified by the identifiers, and receives the nucleic acid sequence data sent from the second database in response to the instruction, to store the data in the memory.

According to an embodiment, in step (b-2), nucleic acid sequence data corresponding to the target nucleic acid molecule are selectively retrieved in the nucleic acid sequence data specified by the identifiers.

The nucleic acid record retrieved by the identifier may contain only nucleic acid sequence data corresponding to the target gene as a target nucleic acid molecule, but in many cases, the nucleic acid record may contain not only the sequence for the target gene but also nucleic acid sequence data for other genes. Furthermore, depending on the purpose of retrieving a target nucleic acid sequence data set for the target nucleic acid molecule, only the nucleic acid sequence data corresponding to a part of the target nucleic acid molecule may need to be retrieved. Therefore, the entirety or a part of the nucleic acid records specified by the identifiers are retrieved, and then nucleic acid sequence data corresponding to the target nucleic acid molecule suitable for the purpose may be selectively retrieved therefrom.

According to an embodiment of the present invention, step (b-2) may include the following steps: (b-2-1) retrieving nucleic acid records specified by the identifiers; and (b-2-2) retrieving nucleic acid sequence data corresponding to the target nucleic acid molecule from the nucleic acid records.

According to an embodiment, in step (b-2-2), nucleic acid sequence data corresponding to the target nucleic acid molecule and identification information of the nucleic acid sequence data may be selectively retrieved from the nucleic acid records.

As used herein, the expression “selectively retrieving” refers to retrieving only necessary nucleic acid sequence data from the nucleic acid sequence data in the nucleic acid record.

When the nucleic acid sequence data contained in the nucleic acid record include a plurality of dividable nucleic acid sequence data, the corresponding nucleic acid record includes multiple sub-records. As used herein, the term “sub-record” refers to a data group unit containing nucleic acid sequence data and/or specifications thereof dividable within one nucleic acid record. Each sub-record contains location information about the nucleic acid sequence data corresponding to each sub-record and specifications containing a description of the nucleic acid sequence data corresponding to each sub-record.

As used herein, the term “dividable nucleic acid sequence data” refers to each of two or more nucleic acid sequences and specifications thereof when one nucleic acid record contains the two or more nucleic acid sequences that may be recognized as being physically or functionally different. For example, when all of nucleic acid sequences for multiple genes encoding different proteins are included in one piece of nucleic acid sequence data in the nucleic acid record, the nucleic acid sequence data may be divided into parts corresponding to respective genes.

In order to selectively retrieve desired nucleic acid sequence data from one nucleic acid record containing a plurality of dividable nucleic acid sequence data, it is determined whether the corresponding sub-record is a valid sub-record, on the basis of the descriptor (specifically, specifications) included in each sub-record in the nucleic acid record. As used herein, the term “valid sub-record” refers to a sub-record that contains nucleic acid sequence data to be retrieved or location information thereof.

The descriptor (specifically, specifications) included in each sub-record refers to a field in which information about nucleic acid sequence data containing the location information thereof is described in each sub-record. The specifications may include, for example, a name of a gene represented by the corresponding sub-record, information about a protein produced from a gene represented by the corresponding sub-record (e.g., protein name and identifier of protein), the note by a nucleic acid record provider, amino acid sequence information, and the like.

The present inventors have identified that the retrieved synonyms are described in some of the specifications of the sub-record, and the frequency of descriptions of the retrieved synonym and the accuracy thereof are different between specifications. Therefore, it was found that one of the most efficient schemes for securing target nucleic acid sequence data is to select some of the specifications, assigning priority thereto, and then sequentially determining whether the retrieved synonym is described in the corresponding specification.

The present inventors compared a gene included in the nucleic acid sequence data represented by each sub-record and the data in specifications of the corresponding sub-record, and as a result, identified that the retrieved synonym is most frequently described in the specification with respect to a gene name, and next in the specification with respect to protein information and the specification with respect to the note by nucleic acid record provider, in that order. Therefore, the present inventors established that checking whether the retrieved synonym is described in the specifications in the above order to thereby determine a valid sub-record and retrieving nucleic acid sequence data of the determined valid sub-record and identification information thereof is a most efficient scheme to accurately and selectively secure desired nucleic acid sequence data.

According to an embodiment of the present invention, the step of selectively retrieving nucleic acid sequence data corresponding to the target nucleic acid molecule and identification information of the nucleic acid sequence data from the nucleic acid records may include the following steps:
(b-2-2-1) determining, as a valid sub-record, a sub-record in which the synonym is recorded in a first specification that is predetermined, among one or more sub-records in each of the nucleic acid records;
(b-2-2-2) determining, as a valid sub-record, a sub-record in which the synonym is recorded in a second specification, when there is no valid sub-record determined by the first specification in the nucleic acid record;
(b-2-2-3) determining, as a valid sub-record, a sub-record in which the synonym is recorded in a third specification, when there is no valid sub-record determined by the second specification in the nucleic acid record; and
(b-2-2-4) retrieving nucleic acid sequence data corresponding to the determined one valid sub-record and identification information thereof.

According to another embodiment of the present invention, the step of selectively retrieving nucleic acid sequence data corresponding to the target nucleic acid molecule and identification information of the nucleic acid sequence data from the nucleic acid records may include the following steps:
(b-2-2-1) determining, as a valid sub-record, a sub-record in which the synonym is recorded in first and second specifications that are predetermined, among one or more sub-records in each of the nucleic acid records;
(b-2-2-2) determining, as a valid sub-record, a sub-record in which the synonym is recorded in a third specification, when there is no valid sub-record determined by the first and second specifications in the nucleic acid record;
and (b-2-2-3) retrieving nucleic acid sequence data for the determined one valid sub-record and identification information thereof.

According to an embodiment of the present invention, the first specification may be a specification with respect to a name of a gene of the nucleic acid sequence in the sub-record; the second specification may be a specification with respect to the information of a protein produced from the gene; and the third specification may be a specification with respect to the note by a gene information provider.

According to still another embodiment of the present invention, the step of selectively retrieving nucleic acid sequence data corresponding to the target nucleic acid molecule and identification information of the nucleic acid sequence data from the nucleic acid records may include the following steps:
(b-2-2-1) determining, as a valid sub-record, a sub-record in which the name of the target nucleic acid and/or the synonym thereof is recorded in a first specification that is predetermined with respect to a name of a gene and the name of the protein of the target nucleic acid molecule and/or the synonym thereof is recorded in a second specification with respect to information of a protein, among one or more sub-records in each of the nucleic acid records;
(b-2-2-2) determining, as a valid sub-record, a sub-record in which at least one of the name of the target nucleic acid molecule and the name of the protein of the target nucleic acid molecule and the synonym thereof is recorded in a third specification with respect to the note by a gene information provider, when there is no valid sub-record determined by the first and second specifications in the nucleic acid record; and
(b-2-2-3) retrieving nucleic acid sequence data for the determined one valid sub-record and identification information thereof.

According to the present embodiment, when, in the descriptor of the nucleic acid record, the name of the gene described in the first specification with respect to a name of a gene is identical to the name of the target nucleic acid molecule received in step (a) and/or the synonym for the target nucleic molecule retrieved in step (a); and the name of the protein described in the second specification with respect to information of a protein is identical to the name of the protein received in step (a) and/or the synonym of the name of the protein retrieved in step (a), nucleic acid sequence data corresponding to the target nucleic acid molecule and identification information of the nucleic acid sequence data can be selectively retrieved from the nucleic acid records.

Therefore, when nucleic acid sequence data are retrieved in which at least one of the name of the target nucleic acid molecule and the retrieved synonyms are described in the name of the gene, the information of the gene, and the note by the gene information provider among the descriptor in a nucleic acid record, a target nucleic acid sequence that is more suitable to design an oligonucleotide can be retrieved.

According to an embodiment of the present invention, the method may further include, after step (b-2-2), step (b-2-2A) of, when a plurality of nucleic acid sequence data are retrieved from one nucleic acid record in step (b-2-2), merging nucleic acid sequence data partially or entirely overlapping each other, among the plurality of nucleic acid sequence data, to provide the nucleic acid sequence data.

For example, the presence of a plurality of nucleic acid sequence data for one target nucleic acid molecule in one nucleic acid record corresponds to a case where each multiple nucleic acid sequence data is a partial sequence of the target nucleic acid molecule. In other words, each of the plurality of nucleic acid sequence data does not independently encode the target nucleic acid molecule-encoding protein, but all of the plurality of nucleic acid sequence data encode one target nucleic acid molecule-encoding protein. In such a case, it is not appropriate to use each of the plurality of nucleic acid sequence data as a target nucleic acid sequence for the target nucleic acid molecule, and it is preferable to handle, as a target nucleic acid sequence for the target nucleic acid molecule, one nucleic acid sequence obtained by merging the plurality of nucleic acid sequence data. For example, when, as for the same gene, the location information described in the descriptor gene includes 1 to 10 and the location information described in the descriptor CDS includes 2 to 8, both the nucleic acid sequence data corresponding to location information 1 to 10 and the nucleic acid sequence data corresponding to location information 2 to 8 may be retrieved according to the present invention. When such two nucleic acid sequence data are provided as a nucleic acid sequence data set for the design of an oligonucleotide, two nucleic acid sequence data are provided even though they correspond to a single nucleic acid sequence having the same identifier, and thus, for example, an oligonucleotide designed by location information 2 to 8 has a problem of having a target-coverage not being 1 but 2. In order to solve such a problem, only one nucleic acid sequence data corresponding to location information 1 to location information 10 is provided by merging overlapping portions of the nucleic acid sequences having the same identifier.

As used herein, the term “target-coverage” refers to a proportion of multiple nucleic acid sequences for a target nucleic acid molecule, which match a sequence of an oligonucleotide (specifically, 100% matching, at least 95% matching, at least 90% matching, or the like).

According to a more specific embodiment of the present invention, the merging in steps (b-2-2A) is performed by a method including the following steps:
(b-2-2A-1) retrieving location information about each of the plurality of nucleic acid sequence data corresponding to the target nucleic acid molecule in the one nucleic acid record (specifically, start-point information and end-point information);
(b-2-2A-2) analyzing the sequence location information to select nucleic acid sequence data, which partially or entirely overlap each other, among the plurality of nucleic acid sequence data; and
(b-2-2A-3) creating new nucleic acid sequence data including all of the selected nucleic acid sequence data.

As described above, one nucleic acid sequence data obtained by merging overlapping partially or entirely nucleic acid sequence data is provided, thereby reducing a probability of statistical errors in the analysis based on a target nucleic acid sequence data set, which occur when target nucleic acid sequence data of a part of the target nucleic acid molecule are recognized as target nucleic acid sequence data for each independent target nucleic acid molecule.

According to an embodiment of the present invention, the method further includes, between steps (b) and (c), the following steps:
(b-3) sorting the retrieved nucleic acid sequence data according to the biosample identification to select nucleic acid sequence data having the same biosample identification;
(b-4) sorting the selected nucleic acid sequence data so as to satisfy at least one of the following sort criteria;
(b-5) selecting a nucleic acid sequence of the highest-ranked nucleic acid sequence data among the sorted nucleic acid sequence data; and
(b-6) deleting nucleic acid sequence data except for the highest-ranked nucleic acid sequence data from the retrieved nucleic acid sequence data, wherein the sort criteria include the following:
(i) sorting the selected nucleic acid sequence data according to the assembly level, wherein as for the assembly level, complete genome, chromosome, scaffold, and config are ranked higher in that order (that is, the ranking of the complete genome is highest); and
(ii) sorting the selected nucleic acid sequence data according to whether the selected nucleic acid sequence data are included in a reference sequence (RefSeq) database, wherein the ranking is higher when the nucleic acid sequence data are included in the RefSeq database than when not included.

Considering genome sequencing according to the genome project, registration in a database, and checking registered sequence information to transfer the sequence to other databases, the present inventors confirmed that when different accession numbers but the same information of a biosample is given to respective nucleic acid sequences, the assembly levels of the genomic sequences are different according to the progress of genome sequencing and the storage databases therefor are different, and the present inventors, by using such characteristics, intend to delete nucleic acid sequence data other than the nucleic acid sequence that has reached the latest genome sequencing from the nucleic acid sequence data retrieved in step (b).

Specifically, when intending to register information about the nucleotide sequence in the database, a person who has established gene and protein information of a genomic sequence under the genome project inputs a bio-project and a biosample associated with the retrieval information of the genomic sequence, and the bio-project and the biosample have a unique number with a combination of letters and numbers.

During the whole genome sequencing according to the genome project, the genomic sequence is fragmented into some sequences, and it is checked whether the sequences are a gene encoding a protein and what functions the protein has, and then some fragmented sequences are merged, and in this procedure, the nucleic acid sequences have assembly levels in the order of config, scaffold, chromosome, and complete genome. That is, the order of the assembly level indicates the progression degree of the genomic sequencing (the assembly level of the complete genome is highest).

When nucleic acid sequences have assembly levels of chromosome and complete genome with the progress of genome sequencing of the genomic sequence, the nucleic acid sequence data are transferred from the initially registered database (e.g., NCBI GenBank) to another database (e.g., NCBI RefSeq), and nucleic acid sequence data having a lower assembly level are deleted from the initially registered database.

However, the nucleic acid sequence data may be present without being deleted from NCBI GenBank in spite of the transfer to the NCBI RefSeq database.

These processes for the genomic sequence are present as information about the nucleic acid sequence data in the descriptor in the nucleic acid record, and in the present embodiment, the present inventors use such information to delete redundant sequences.

For example, when NCBI GenBank and RefSeq databases are included in the second database used in the present invention, if nucleic acid records associated with assembly levels of config, scaffold, and chromosome of the organism of interest are still present in the GenBank database of NCBI even though the nucleic acid sequence data of complete genome are present in the RefSeq database due to the completion of sequencing for the genomic sequence of an organism of interest, nucleic acid sequence data with the four types of assembly levels are included in the nucleic acid sequence data retrieved according to the present invention.

When the nucleic acid sequence data with the four types of assembly levels are provided to design an oligonucleotide, the target-coverage may increase by 4 times even through the nucleic acid sequences are identical. Moreover, when in the genome sequencing procedure, sequence information is revised at the chromosome level and the revised sequence information is reflected at the complete genome level and then an oligonucleotide is designed from the revised sequence information, degenerate bases need to be introduced at the position where the sequence information is revised.

In such a case, a redundant sequence may be deleted to prevent the above-described problem by using the fact that the nucleic acid sequence data corresponding to the four types of assembly levels are nucleic acid sequence data for the same organism having information about the same biosample.

The biosample identification in step (b-3) indicates a unique number of a biosample. If the nucleic acid sequence data is included in the reference sequence (RefSeq) database in (ii) above, a unique number is assigned to the nucleic acid record.

The deletion of redundant sequences according to the present embodiment can make improvements in the target-coverage in the design of an oligonucleotide and the time and cost associated with the introduction of degenerate bases.

### Step (c): Selecting Taxonomic Representative Sequence (130)

Then, the method of the present invention includes step (c) of sorting the retrieved nucleic acid sequence data according to the taxonomic name and/or taxonomic ID and selecting taxonomic representative sequences among nucleic acid sequence data having the same taxonomic name and/or taxonomic ID.

As used herein, the term “taxonomic name” refers to a scientific name of an organism classified according to the biological taxonomic system, and the term “taxonomic ID” refers to a number that is assigned to the taxonomic name. The taxonomic ID is linked to, for example, organism information of the nucleic acid record retrieved from NCBI and a taxonomy database of NCBI, and thus can be seen through the taxonomy viewer and can also be seen on the taxon field of the nucleic acid record retrieved from the NCBI.

The retrieved nucleic acid sequence data are sorted according to the taxonomic name and/or the taxonomic ID.

Among the nucleic acid sequence data sorted according to the taxonomic name and/or taxonomic ID, nucleic acid sequence data having the same taxonomic name and/or taxonomic ID are sorted, and taxonomic representative sequences are selected therefrom.

According to an embodiment of the present invention, the selecting of the taxonomic representative sequence in step (c) is performed by a method including the following steps:
(c-1) sorting the nucleic acid sequence data having the same taxonomic name and/or taxonomic ID so as to satisfy at least one of the following predetermined sort criteria; and
(c-2) selecting, as the taxonomic representative sequence, a nucleic acid sequence of the highest-ranked nucleic acid sequence data among the sorted nucleic acid sequence data, wherein the predetermined sort criteria include the following:
(i) sorting according to the assembly level of the nucleic acid sequence data, wherein as for the assembly level, complete genome, chromosome, scaffold, and config are ranked higher in that order (that is, the ranking of the complete genome is highest);
(ii) sorting according to whether the nucleic acid sequence data are included in a reference sequence (RefSeq) database, wherein the ranking is higher when the nucleic acid sequence data are included in the RefSeq database than when not included;
(iii) sorting according to whether a name of a nucleic acid molecule described in a descriptor of a nucleic acid record containing the nucleic acid sequence data is identical to at least one of the received name of the target nucleic acid and the retrieved synonyms, wherein the ranking is higher when identical than when not identical;
(iv) sorting according to the length of the nucleic acid sequence data, wherein the longer the length, the higher the ranking;
(v) sorting according to whether there is description in a host of a descriptor of a nucleic acid record containing the nucleic acid sequence data, wherein the ranking is higher when a host of interest for the organism of interest is described in the host than when not described, and higher when an organism different from the organism of interest are not described in the host than when described;
(vi) sorting according to the registration date or revision date of a nucleic acid record containing the nucleic acid sequence data, wherein the more recent the registration date or revision date, the higher the ranking; and
(vii) sorting according to the alphabetical order in accession number of the nucleic acid sequence data, wherein the earlier the alphabetical order of the accession number, the higher the ranking.

According to the present embodiment, the nucleic acid sequence data having the same taxonomic name and/or taxonomic ID are sorted to satisfy sort criteria considering at least one criterion (specifically, sort criterion (i)), specifically at least two, more specifically at least three, at least four, at least five, or at least six, and most specifically seven criteria.

According to an embodiment of the present invention, the at least two sort criteria are different in criticality, and the method of the present invention further includes a step of sorting nucleic acid sequence data to satisfy at least two sort criteria considering the criticality.

There may be largely two methods for sorting nucleic acid sequence data having the same taxonomic name and/or taxonomic ID to select the taxonomic representative sequences.

According to the first method, the at least two sort criteria are different in criticality, and the nucleic acid sequence data may be sorted to satisfy the sort criterion with the highest criticality (e.g., sort criterion (i)).

If there are a plurality of nucleic acid sequence data satisfying the sort criterion with the highest criticality, the nucleic acid sequence data may be sorted to satisfy the next-ranked sort criterion.

For example, if the criticality in sort criterion is the order of sort criteria (i), (ii), (iii), (iv), (v), (vi), and (vii) and three nucleic acid sequence data satisfy sort criterion (i), the three nucleic acid sequence data are sorted according to sort criterion (ii). If three nucleic acid sequence data satisfy sort criterion (ii), the nucleic acid sequence data are sorted to satisfy sort criterion (iii).

According to the second method, different weights are assigned to the sort criteria and scores are assigned to values (or value ranges) in each of the sort criteria, and thus, the total score of each of the nucleic acid sequence data may be obtained considering priority therefor. The nucleic acid sequence data may be sorted in consideration of the calculated total score, and the highest-ranked nucleic acid sequence data may be selected as a taxonomic representative sequence through the ranking of total scores.

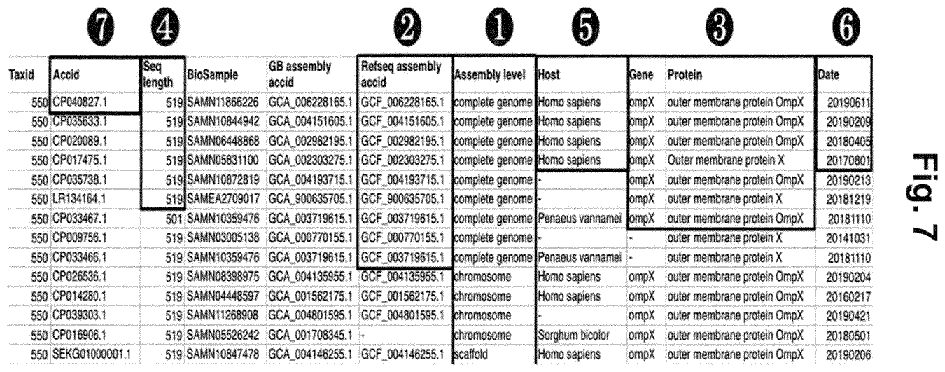

FIG. 7 shows that nucleic acid sequence data specified by identifiers retrieved using the name of a target nucleic acid molecule (ompX) of an organism of interest (Enterobacter cloacae complex) and a synonym thereof (outer membrane protein) are retrieved, and among nucleic acid sequence data having the same taxonomic ID, the nucleic acid sequence data are sorted according to the above-described sort criteria, and a taxonomic representative sequence is selected by the above-described first method.

Referring to FIG. 7, the retrieved nucleic acid sequence data are identical in light of taxonomic ID (Taxid) being 550. The criticality in sort criterion is the order of sort criteria (i), (ii), (iii), (iv), (v), (vi), and (vii). First, sorting is conducted according to the assembly level of a nucleic acid sequence in sort criterion (i) (the higher the assembly level, the higher the ranking); sorting is conducted according to whether the selected nucleic acid sequence data are included in a reference sequence (RefSeq) database in sort criterion (ii) (the nucleic acid sequence data included in the RefSeq database has a unique number); sorting is conducted according to whether the name of a nucleic acid molecule described in the descriptor of the nucleic acid record is identical to at least one of the received name of the target nucleic acid and the retrieved synonyms in sort criterion (iii) (the ranking is higher in the order of when the nucleic acid molecule name and the protein name described in the descriptor of the nucleic acid record are identical thereto, when the protein name is identical thereto, and when the nucleic acid molecule name is identical thereto); sorting is conducted according to the length of a nucleic acid sequence in sort criterion (iv) (the longer the length, the higher the ranking); sorting is conducted according to whether a host is described in the descriptor of the nucleic acid record in sort criterion (v) (the ranking is higher in the order of when Homo sapience is described in the host, when an organism of interest is not described in the host, and when an organism excluding the Homo sapience is described in the host); sorting is conducted according to the registration date or revision date of a nucleic acid record in sort criterion (vi) (the more recent the registration date or revision date, the higher the ranking); and sorting is conducted according to the alphabet of accession number of a nucleic acid sequence data in sort criterion (vii) (the earlier the alphabetic order of the accession number, the higher the ranking).

As a result, nucleic acid sequence data having Accession No. CP040827.1 is ranked highest, and such a nucleic acid sequence with the highest ranking is selected as a taxonomic representative sequence.

When only nucleic acid sequence data having the same scientific name (the same taxonomic name or taxonomic ID) as the organism of interest are included in the nucleic acid sequence data retrieved in step (b), one taxonomic representative sequence may be selected.

According to an embodiment of the present invention, at least one taxonomic representative sequence may be selected from the retrieved nucleic acid sequence data.

When not only nucleic acid sequence data having the same scientific name (the same taxonomic name or taxonomic ID) as the organism of interest but also nucleic acid sequence data of organisms belonging to a superclass or subclass of the organism of interest and organisms having a different scientific name (taxonomic name or taxonomic ID) in terms of the biological taxonomic system are included in the nucleic acid sequence data retrieved in step (b), multiple taxonomic representative sequences may be selected. That is, each taxonomic representative sequence is selected for each scientific name (taxonomic name or taxonomic ID) of an organism.

According to an embodiment of the present invention, the taxonomic representative sequences satisfy the following criterion of a predetermined length: within a predetermined range of an intermediate value of lengths of nucleic acid sequences having assembly levels of complete genome and/or chromosome in the nucleic acid sequence data retrieved in step (b). The predetermined range is not particularly limited, but may be, for example, ±2%, ±4%, ±5%, ±10%, ±15%, ±20%, ±25%, or ±30% of the intermediate value (bp, mer, or nucleotide length).

### Step (d): Selecting Group Representative Sequence (140)

Then, the method of the present invention further includes step (d) of grouping the selected taxonomic representative sequences according to homology and selecting a group representative sequence for each group.

Since the retrieved synonyms and the nucleic acid sequence data secured using the synonyms are searched on the basis of names, sequences having low sequence identity therebetween due to large variations between nucleic acid sequences may also be retrieved. However, when nucleic acid sequence data are recorded in the second database (specifically, nucleotide database) as follows: the name of the nucleic acid molecule is not yet settled or a sequence is registered with a name that is neither a known name of the target nucleic acid molecule nor a synonym thereof due to the mistake of the registerer or the like, such a sequence may not be retrieved even though the corresponding sequence is substantially a target nucleic acid sequence for the target nucleic acid molecule.

To compensate for this problem, the conventional method (WO2019/212238) provided a nucleic acid sequence data set used to design an oligonucleotide by retrieving nucleic acid sequence data on the basis of synonyms for a target nucleic acid molecule of an organism of interest, sorting the retrieved nucleic acid sequence data according to the sequence length, determining as a representative sequence a nucleic acid sequence having the longest sequence length, grouping the representative sequence and the retrieved nucleic acid sequence data according to the homology, determining as a group representative sequence nucleic acid sequence data having the longest sequence length in each group, adding nucleic acid sequence data having a homology of a predetermined value or more with the determined group representative sequence to supplement the nucleic acid sequence data to each group, and then adding the nucleic acid sequence data supplemented through the group representative sequence to the retrieved nucleic acid sequence data based on the synonyms.

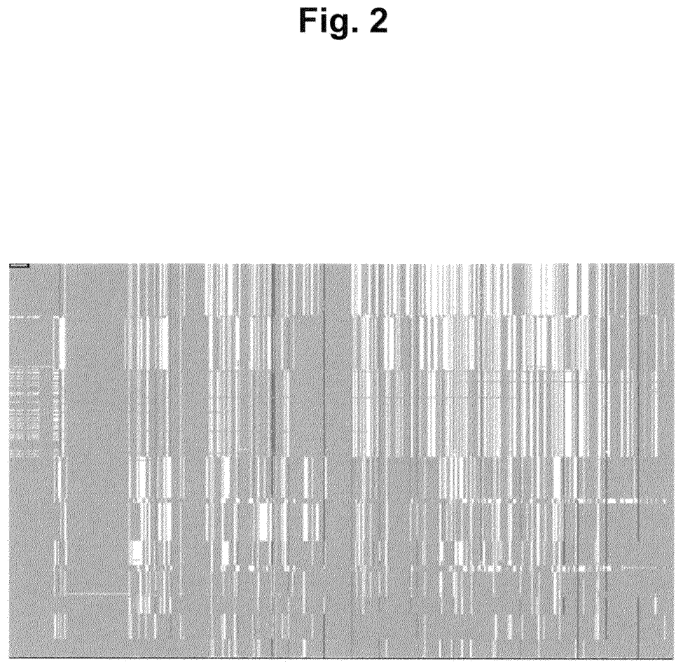

As a result of providing the nucleic acid sequence data set, obtained through such a procedure, in the form of an alignment file, the alignment results of a plurality of nucleic acid sequence data were not properly formed as can be confirmed in FIG. 2, and in order to use the nucleic acid sequence data set in the design of an oligonucleotide, analysts needed to review errors of registration of group representative sequences from the alignment results.

Moreover, as a result of reviewing the alignment results, the present inventors confirmed that the alignment of the retrieved nucleic acid sequence data was not properly formed due to the group representative sequence selected by the above-described method.

Therefore, the present inventors verified that as a result of sorting the sequences retrieved by synonyms according to the taxonomic name and/or taxonomic ID; selecting, as a taxonomic representative sequence, nucleic acid sequence data having the same taxonomic name and/or taxonomic ID; grouping the taxonomic representative sequences according to homology; and selecting a group representative sequence for each group therefrom, the alignment results of the nucleic acid sequence data retrieved therefrom were properly formed.

According to the present invention, for selection of group representative sequences, the selected taxonomic representative sequences were grouped according to homology.

As used herein, the term “homology” refers to relative, positional, and structural similarity or identity between two or more nucleic acid sequences. The homology may be expressed by quantifying the similarity or identity between two nucleic acid sequences, and specifically, may be expressed as percentage.

Specifically, the homology may be identity or similarity. As used herein, the term “identity” is determined by whether bases at specific positions of two sequences to be compared are identical to each other. As used herein, the term “similarity” is determined by differentiating bases having the same characteristics, bases having different but similar characteristics, or bases having different characteristics, in consideration of characteristics of bases at specific positions of two sequences to be compared and then quantitatively converting these bases.

As used herein, the term “identity” and “similarity” used while expressing homology can be used interchangeably, and specifically, the homology may be expressed as identity.

For example, when nucleotides of two nucleic acid sequences are completely identical to each other, the homology therebetween is 100%. When there are non-identical nucleotides between the two nucleic acid sequences, the homology percentage (%) value is reduced. In general, the homology may be obtained by quantifying a degree of the identity between two nucleic acid sequences. The degree of homology may be determined by comparing specific positions of the respective sequences that are aligned with each other for comparison. If there are identical bases at the specific positions of the two sequences to be compared, the two nucleic acid sequences have homology at the corresponding positions. The degree of homology between two sequences may be calculated as a percentage of the number of homologous positions shared by the two sequences.

As used herein, the term “align” or “alignment” refers to a series of techniques for juxtaposing molecular sequences having homology. The calculation of the alignment and homology values of the sequences may be determined by software known in the art, and various methods and algorithms for the alignment are disclosed in Smith and Waterman, Adv. Appl. Math. 2:482(1981); Needleman and Wunsch, J. Mol. Bio. 48:443(1970); Pearson and Lipman, Methods in Mol. Biol. 24: 307-31(1988); Higgins and Sharp, Gene 73:237-44(1988); Higgins and Sharp, CABIOS 5:151-3(1989); Corpet et al., Nuc. Acids Res. 16:10881-90(1988); Huang et al., Comp. Appl. BioSci. 8:155-65(1992) and Pearson et al., Meth. Mol. Biol. 24:307-31(1994). The NCBI Basic Local Alignment Search Tool (Altschul et al., J. Mol. Biol. 215:403-10(1990)) is accessible by National Center for Biological Information (NCBI) or the like, and on the Internet, may be used in conjunction with sequence analysis programs, such as, blastn, blastp, blastx, tblastn, and tblastx. BLAST is available at http://www.ncbi.nlm.nih.gov/BLAST/. A comparison method of sequence similarity using this program may be found at http://www.ncbi.nlm.nih.gov/BLAST/blast_help.html.

When the number of taxonomic representative sequences selected in step (c) is one, the selected taxonomic representative sequence becomes a group representative sequence.

When the number of taxonomic representative sequences selected in step (c) is at least two, the selected taxonomic representative sequences are grouped according to homology, and a group representative sequence is selected from each group.

The group representative sequence is a sequence representative of the group, and the homology according which the selected taxonomic representative sequences are grouped is not particularly limited, but a homology reference value may be determined in advance and applied.

The homology reference value may be determined according to the characteristics of the target nucleic acid molecule. For example, the homology reference value may vary depending on the range of the target nucleic acid molecule. For example, the homology reference value may vary depending on whether the target nucleic acid molecule is specific to a specific species or a specific subspecies. Alternatively, the homology reference value may vary depending on the degree of variation of the target nucleic acid molecule as an object of detection. The homology reference value may be specifically at least 10%, at least 20%, at least 30%, at least 40%, at least 50%, at least 60%, at least 70%, at least 80%, or at least 90%, or 100%, and more specifically, at least 50%, at least 60%, at least 70%, at least 80%, or at least 90%, or 100%. Alternatively, the homology reference value may be determined in a range of 70% to 100%, 80% to 100%, or 90% to 100%.

The selected taxonomic representative sequences were grouped according to the homology, and the group representative sequences may be selected by a known program (e.g., UCLUST).

According to an embodiment of the present invention, the selecting of the group representative sequence in step (d) is performed by a method including the following steps:
(d-1) sorting the selected taxonomic representative sequences so as to satisfy at least one of the following predetermined sort criteria;
(d-2) selecting the highest-ranked taxonomic representative sequence among the sorted taxonomic representative sequences; and
(d-3) grouping taxonomic representative sequences having a homology of a predetermined value or more with the highest-ranked taxonomic representative sequence and selecting the highest-ranked taxonomic representative sequence as the group representative sequence in each group, wherein the sort criteria include the following:
(i) sorting according to the assembly level of the selected taxonomic representative sequence, wherein as for the assembly level, complete genome, chromosome, scaffold, and config are ranked higher in that order;
(ii) the number of nucleic acid sequence data having the same taxonomic name and/or taxonomic ID as the selected taxonomic representative sequence, wherein the larger the number, the higher the ranking;
(iii) sorting according to whether there is a description in a host of a descriptor of a nucleic acid record containing the selected taxonomic representative sequence, wherein the ranking is higher when a host of interest for the organism of interest is described in the host than when not described, and higher when an organism different from the host of interest is not described in the host than when described; and
(iv) sorting according to the alphabetic order of accession number in the selected taxonomic representative sequence, wherein the earlier the alphabet order of the accession number, the higher the ranking.

In the embodiment regarding the selection of taxonomic representative sequences, the description of criticality and weight in the description of the sort criteria of nucleic acid sequence data having the same taxonomic name and/or taxonomic ID can be equally applied to the sort criteria associated with the selection of the group representative sequences.

The description of the homology reference values used while describing the grouping of the taxonomic representative sequences can be equally applied to a homology of a predetermined value or more in step (d-3).

According to step (d-1) of the present embodiment, the selected taxonomic representative sequences are sorted to satisfy sort criteria considering at least one (specifically, sort criterion (i)), specifically at least two, more specifically at least three, and most specifically four rankings.

According to an embodiment of the present invention, the at least two sort criteria are different in criticality, and the method of the present invention further includes a step of sorting taxonomic representative sequences to satisfy a least two sort criteria considering the criticality.

According to step (d-2) of the present embodiment, the highest-ranked taxonomic representative sequence is selected from the sorted taxonomic representative sequences.

According to step (d-3) of the present embodiment, taxonomic representative sequences having a homology of a predetermined value or more with the highest-ranked taxonomic representative sequence are grouped, and the highest-ranked taxonomic representative sequence is selected as a group representative sequence.

Due to the difference in homology between the selected taxonomic representative sequences, the selected taxonomic representative sequences are grouped into multiple groups, and thus multiple group representative sequences may be selected.

When the plurality of group representative sequences are selected, the homology between the group representative sequences is lower than the homology reference value at the time of grouping. When the homology between the representative sequences of the two groups is equal to or greater than the homology reference value, the two group representative sequences need to belong to the same group.

When multiple group representative sequences are selected, the multiple group representative sequences may be selected to satisfy the following conditions:
(i) Each group representative sequence should have a homology of a homology reference value or more with taxonomic representative sequences of its own group; and
(ii) The homology between group representative sequences needs to have a homology of less than the homology reference value of (i) above.

The description of the homology reference value used while describing the grouping of the taxonomic representative sequences can be equally applied to a homology of the homology reference value or more in (i).

When multiple taxonomic representative sequences having a difference in homology are included so that multiple group representative sequences are selected, the following method may be additionally performed.

According to a more specific embodiment of the present invention, the method further includes the following steps: (d-4) selecting the highest-ranked taxonomic representative sequence from remaining taxonomic representative sequences except for the group representative sequence and taxonomic representative sequences included in a group of the group representative sequence from the selected taxonomic representative sequence; (d-5) replacing the highest-ranked taxonomic representative sequence in (d-4) with the highest-ranked taxonomic representative sequence in step (d-3) and performing step (d-3); and repeating steps (d-4) and (d-5) when there are taxonomic representative sequences that are to be additionally grouped.

### Step (e): Providing Nucleic Acid Sequence Data Set for the Design of an Oligonucleotide (150)

Last, the method of the present invention includes step (e) of retrieving nucleic acid sequence data having a homology of a predetermined value or more with the group representative sequence and providing the retrieved nucleic acid sequence data as a nucleic acid sequence data set for the design of an oligonucleotide.

According to the present invention, nucleic acid sequence data are retrieved by the name of the target nucleic acid molecule of the organism of interest and/or synonyms thereof; taxonomic representative sequences and a group representative sequence are selected therefrom; and nucleic acid sequence data having a homology of a predetermined value or more with the group representative sequence are retrieved and provided as a nucleic acid sequence data set for the design of an oligonucleotide.

When the number of group representative sequences is two or more, nucleic acid sequence data having a homology of a predetermined value or more with each of the group representative sequences are retrieved, respectively, and provided as a nucleic acid sequence data set for the design of an oligonucleotide.

The predetermined value or more in the homology is not particularly limited, but for example, may be a homology of 10% or more, 20% or more, 30% or more, 40% or more, 50% or more, 60% or more, 70% or more, 80% or more, 90% or more, or 100%. Specifically, the homology may be a homology of 40% or more, 50% or more, 60% or more, 70% or more, 80% or more, or 90% or more, or 100%, and more specifically, 40% or more, 50% or more, 60% or more, 70% or more, or 80% or more. Alternatively, the homology may be selected within a range of 10 to 100%, more specifically, within a range of 40% to 100%, and still more specifically, in a range of 40%, 50%, 60%, 70%, or 80%.

According to an embodiment of the present invention, the nucleic acid sequence data having a homology of a predetermined value or more are retrieved from a second database.

Since the description of the second database in step (b) can be equally applied to the present step, the common descriptions among them are omitted in order to avoid undue redundancy leading to the complexity of this specification.

The retrieving may be performed using software (e.g., BLAST) known in the art.

The providing may be performed by various data provision methods known in the art. For example, the data set may be provided by a method in which the data are exposed to a user through an output device or a display device to enable the user to directly recognize the content of the data; the data set may be provided to a user by a method in which the data are stored, through a storage device, in a data storage medium desired by the user; or the data set may be provided by a method in which the data are transmitted to a device intended by the user via a network device capable of wired or wireless data transmission.

According to an embodiment of the present invention, the provided nucleic acid sequence data set for the design of an oligonucleotide is the nucleic acid sequence data set, a list of nucleic acid sequence data sets containing information about the nucleic acid sequence data, and/or the alignment result of aligning the nucleic acid sequence data set.

The information about the nucleic acid sequence data indicates information including all the information described in the nucleic acid record containing the nucleic acid sequence data, and includes, for example, accession number of the nucleic acid sequence data, group number of a group including the nucleic acid sequence data, location information of a gene in the nucleic acid sequence data, organism name (or taxonomic name), taxonomic ID, gene name, protein name, homology information, and the like.

The alignment result of aligning the nucleic acid sequence data indicates the alignment result of aligning using various alignment programs known in the art, and the alignment result is provided in the form of a file provided by the alignment programs.

Since the description of various methods and algorithms for the alignment in step (c) can be equally applied to this step, the common descriptions among them are omitted in order to avoid undue redundancy leading to the complexity of this specification.

According to an embodiment of the present invention, the method further includes, after step (e), the following step:
(e-1) sorting the provided nucleic acid sequence data set for the design according to the biosample identification to select nucleic acid sequence data having same biosample identification;
(e-2) sorting the selected nucleic acid sequence data so as to satisfy at least one of the following sort criteria;
(e-3) selecting a nucleic acid sequence of the highest-ranked nucleic acid sequence data among the sorted nucleic acid sequence data; and
(e-4) deleting nucleic acid sequence data except for the highest-ranked nucleic acid sequence data from the nucleic acid sequence data set for the design, wherein the sort criteria include the following:
(i) sorting nucleic acid sequences included in the provided nucleic acid sequence data set for the design according to the assembly level, wherein as for the assembly level, complete genome, chromosome, scaffold, and config are ranked higher in that order (that is, the ranking of the complete genome is highest); and
(ii) sorting whether nucleic acid sequences included in the provided nucleic acid sequence data set for the design are included in a reference sequence (RefSeq) database, wherein the ranking is higher when the nucleic acid sequence data are included in the RefSeq database than when not included.

Since the description of the steps (b-3) to (b-6) performed between steps (b) and (c) can be equally applied to the present embodiment, the common descriptions among them are omitted in order to avoid undue redundancy leading to the complexity of this specification.

The deletion of the redundant sequence carried out in steps (b-3) to (b-6) is performed on nucleic acid sequence data retrieved by the name of the target nucleic acid molecule for the organism of interest and/or synonyms thereof, but the deletion of the redundant sequence in the present embodiment is performed on the nucleic acid sequence data set for the design of an oligonucleotide provided in step (e).

In the present embodiment, as for the deletion of a redundant sequence, the redundant sequence may be deleted from the nucleic acid sequence data set provided in step (f) to be described later or the non-target nucleic acid sequence data set provided in step (g) to be described later, which are targets for deletion of a redundant sequence.

The deletion of a redundant sequence according to the present embodiment can make improvements in the target-coverage in the design of an oligonucleotide and the time and cost associated with the introduction of degenerate bases.

The nucleic acid sequence data set for the design of an oligonucleotide provided by the method of the present invention comprise nucleic acid sequence data associated with the received organism of interest and/or received nucleic acid sequence data not associated with the received organism of interest.

Herein, the nucleic acid sequence data set associated with an organism of interest in the nucleic acid sequence data set for the design of an oligonucleotide indicates a target nucleic acid sequence data set for the target nucleic acid molecule, and the nucleic acid sequence data set not associated with an organism of interest in the nucleic acid sequence data set for the design of an oligonucleotide indicates a non-target nucleic acid sequence data set for a non-target nucleic acid molecule.

An oligonucleotide used to amplify or detect a target nucleic acid molecule in an organism of interest need to satisfy the following two design requirements: First, the oligonucleotide should detect multiple target nucleic acid sequences having sequence similarity to the target nucleic acid molecule of the organism of interest with as high a target-coverage as possible. Second, the nucleotide should not detect nucleic acid molecules of organisms other than the organism of interest.

In the nucleic acid sequence data set provided to design an oligonucleotide satisfying these two requirements, a target nucleic acid sequence data set for a target nucleic acid molecule is provided for the first requirement and a non-target nucleic acid sequence data set for a non-target nucleic acid molecule is provided for the second requirement.

### Step (f): Providing Target Nucleic Acid Sequence Data Set for Target Nucleic Acid Molecule

According to an embodiment of the present invention, the method further includes step (f) of providing, as a target nucleic acid sequence data set for the target nucleic acid molecule, nucleic acid sequence data associated with the received organism of interest from the nucleic acid sequence data set provided in step (e).

In the retrieving of the target nucleic acid sequence using the name of the target nucleic acid molecule of the organism of interest and/or synonyms thereof, when the name of the nucleic acid sequence is changed after registration of the nucleic acid sequence, or the information about the nucleic acid sequence is erroneously described or omitted due to the mistake of a nucleic acid sequence registerer, the corresponding nucleic acid sequence may not be retrieved. Thus, the method of providing a set of the nucleic acid sequence data associated with the received organism of interest among nucleic acid sequence data having homology of a predetermined value or more with the group representative sequence may solve the problem of missing the target nucleic acid sequence data for the above reason.

The nucleic acid sequence data associated with the received organism of interest mean nucleic acid sequence data wherein the name of the organism of interest received in the step (a) or synonyms thereof, or the name of an organism belonging to a subclass of the received organism of interest in terms of taxonomy or synonyms thereof is described as an organism associated with the nucleic acid sequence data. However, the nucleic acid sequence data associated with the received organism of interest does not mean nucleic acid sequence data wherein the name of an organism belonging to a superclass of the received organism of interest in terms of taxonomy or synonyms thereof is described as an organism associated with the nucleic acid sequence data.

According to the present embodiment, a target nucleic acid sequence data set for the target nucleic acid molecule can be provided by retrieving nucleic acid sequence data having a homology of at least a predetermined value with the group representative sequence from the nucleotide database and then comparing information about an organism associated with the retrieved sequence and the information about the organism of interest associated with the target nucleic acid molecule.

The information about the organism may be a scientific name or taxonomic name of the organism, or taxonomic ID assigned to the scientific name or taxonomic name of the organism, which is described in a title of a nucleic acid record for each nucleic acid sequence or the organism as a descriptor thereof.

As for the providing, when the information about an organism associated with the retrieved nucleic acid sequence data is identical to the information of the organism of interest associated with the target nucleic acid molecule or the information about an organism belonging to a subclass of the organism of interest associated with the target nucleic acid molecule, the retrieved nucleic acid sequence data may be provided as nucleic acid sequence data included in the target nucleic acid sequence data set for the target nucleic acid molecule.

According to the present embodiment, even sequences, which are not included in the name-based sequence retrieval due to the missing of some synonyms during retrieving synonyms or the erroneous recording of names of nucleic acid molecules at the initial sequence registration, can be retrieved.

As used herein, the term “target nucleic acid sequence” refers to a sequence related to the target nucleic acid molecule, which is a nucleic acid molecule to be finally detected. The target nucleic acid sequence may include the entirety or a part of the nucleic acid sequence corresponding to the target nucleic acid molecule.

The nucleic acid sequences of a common specific gene possessed by a specific organism group may be identical or different between entities. Thus, when the target nucleic acid molecule represents a common specific gene possessed by a specific organism group, the nucleic acid sequence corresponding to the target nucleic acid molecule cannot be settled by one arrangement order of nucleotides. In other words, one target nucleic acid molecule may have a plurality of target nucleic acid sequence data with different arrangement orders of nucleotides.

The target nucleic acid sequence data set refers to a collection of target nucleic acid sequence data. That is, the target nucleic acid sequence data set refers to a collection of information about the arrangement order of nucleotides of the target nucleic acid molecule. As described above, one target nucleic acid molecule may have various target nucleic acid sequence data with different arrangement orders of nucleotides.

Therefore, according to an embodiment of the present invention, the target nucleic acid sequence data set for the target nucleic acid molecule may be a data set containing a plurality of target nucleic acid sequence data.

According to an embodiment of the present invention, the target nucleic acid sequence data set may include nucleic acid sequence data corresponding to a part or the entirety of the target nucleic acid molecule, or variant nucleic acid sequence data for the target nucleic acid molecule.

Since the target nucleic acid sequence data for the target nucleic acid molecule refers to nucleic acid sequence data related to a nucleic acid sequence of the target nucleic acid molecule of an organism of interest, the target nucleic acid sequence data includes all of nucleic acid sequence data consisting of only the entirety or a part of the nucleic acid sequence corresponding to the target nucleic acid molecule and nucleic acid sequence data comprising the entirety or a part of the nucleic acid sequence corresponding to the target nucleic acid molecule of the organism of interest.

The variant nucleic acid sequence for the target nucleic acid molecule refers to a nucleic acid sequence that includes a nucleotide sequence in which at least one nucleotide is substituted, deleted and/or added compared with the target nucleic acid sequence for the target nucleic acid molecule.

According to an embodiment of the present invention, the target nucleic acid sequence data set for the target nucleic acid molecule may be provided including information of alignment with a group representative sequence. More specifically, the target nucleic acid sequence data set for the target nucleic acid molecule may be provided in the form of an alignment file in which a group representative sequence is aligned with the target nucleic acid sequences of the target nucleic acid sequence data set. Through such information of alignment, the target nucleic acid sequence data set for the target nucleic acid molecule can be more effectively used in the design of a desired oligonucleotide.

According to a more specific embodiment of the present invention, the target nucleic acid sequence data set provided in step (f) have a homology of a predetermined value or more with at least one representative sequence of the group representative sequence and the taxonomic representative sequence for the target nucleic acid sequence data set.

According to the present invention, in step (e), nucleic acid sequence data having a homology of at least a predetermined value with the group representative sequence are retrieved; and in step (f), nucleic acid sequence data associated with the organism received in step (a) are provided among the retrieved nucleic acid sequence data and then nucleic acid sequence data having a homology of at least a predetermined value with at least one representative sequence of the group representative sequence and the taxonomic representative sequence for the nucleic acid sequence data can be provided as a target nucleic acid sequence data set. Therefore, the present embodiment may also be expressed by the above-described time-series process.

The predetermined value associated with homology in the present embodiment is larger than the predetermined value associated with homology in step (e), and specifically, the predetermined value associated with homology in the present embodiment may be larger than the predetermined value associated with homology in step (e) by 5%, 10%, 15%, 20%, 25%, 30%, 35%, 40%, 45%, 50%, 55%, or 60%.

In the present embodiment, unlike in step (e), a nucleic acid sequence data set having homology with a group representative sequence and/or a taxonomic representative sequence is additionally provided, as a target nucleic acid sequence data set, to the nucleic acid sequence data associated with the organism of interest in the nucleic acid sequence data set having homology with the group representative sequence.

The reason for considering not only the group representative sequence but also the taxonomic representative sequence in the present embodiment is to retrieve the target nucleic acid sequence data that should be detected by the oligonucleotide, without omission, in the design of an oligonucleotide used to detect the target nucleic acid molecule.

The at least a predetermined value in the homology of the present embodiment may be a homology of at least 70%, at least 80%, at least 90%, at least 95%, at least 96%, at least 97%, at least 98%, or at least 99%, or 100%. The predetermined value may be a value in a predetermined range, and may be 70% to 100%, 80% to 100%, 90% to 100%, or 95% to 100%, but is not limited thereto.

### Step (g): Providing Non-Target Nucleic Acid Sequence Data Set for Non-Target Nucleic Acid Molecule

According to an embodiment of the present invention, the method further includes step (g) of providing, as a non-target nucleic acid sequence data set for a non-target nucleic acid molecule, nucleic acid sequence data not associated with the received organism of interest from the nucleic acid sequence data set provided in step (e).

According to the present embodiment, nucleic acid sequence data not associated with the received organism of interest in the nucleic acid sequence data set having a homology of a predetermined value or more with the group representative sequence retrieved in step (e) is referred to a non-target nucleic acid sequence data set for a non-target nucleic acid molecule.

The present embodiment is performed for the second requirement wherein nucleic acid molecules of other organisms but not the organism of interest should not be detected, of the above-described oligonucleotide designing requirements. Specifically, another challenge in the design of an oligonucleotide for detection of a specific target nucleic acid molecule is that information about nucleic acid molecules that may result in a false-positive error is identified so that such nucleic acid molecules should not be detected.

In order to solve this challenge, in the present embodiment, a non-target nucleic acid sequence data set for non-target nucleic acid molecules that may result in a false-positive error is provided and this set may be used to design an oligonucleotide so that the non-target nucleic acid sequence data are not detected.

The nucleic acid sequence data not associated with the received organism of interest indicate nucleic acid sequence data wherein the name of the organism of interest received in the step (a) or synonyms thereof, or a name of an organism belonging to a subclass of the received organism of interest in terms of taxonomy or synonyms thereof are not described as an organism associated with the nucleic acid sequence data. Therefore, the nucleic acid sequence data not associated with the received organism of interest indicate nucleic acid sequence data wherein a name of an organism belonging to a superclass of the received organism of interest in terms of taxonomy or synonyms thereof, or a name of an organism different from the received organism of interest or synonyms thereof are described as an organism associated with the nucleic acid sequence data.

According to the present embodiment, a non-target nucleic acid sequence data set for a non-target nucleic acid molecule can be provided by retrieving nucleic acid sequence data having a homology of at least a predetermined value with the group representative sequence from the nucleotide database and then comparing information about an organism associated with the retrieved sequence and the information about the organism of interest associated with the target nucleic acid molecule.

Specifically, nucleic acid sequence data having a homology of a predetermined value or more and information about an organism of interest associated therewith are retrieved from the second database, and nucleic acid sequence data wherein the information about the organism in the retrieved nucleic acid sequence data is not included in the organism of interest received in step (a) or an organism pertaining to a subclass thereof may be provided as a non-target nucleic acid sequence for a non-target nucleic acid molecule.

The information about the organism may be a scientific name or taxonomic name of the organism, or taxonomic ID assigned to the scientific name or taxonomic name of the organism, which is described in a title of a nucleic acid record for each nucleic acid sequence or the organism as a descriptor thereof.

As for the providing, when the information about an organism associated with the retrieved nucleic acid sequence data is different from the information of the organism of interest associated with the target nucleic acid molecule or is identical to the information about an organism belonging to a superclass of the organism of interest associated with the target nucleic acid molecule, the nucleic acid sequence data may be provided as nucleic acid sequence data included in the non-target nucleic acid sequence data set for the non-target nucleic acid molecule.

As used herein, the term “non-target nucleic acid molecule” has a contrary concept to the target nucleic acid molecule, and refers to a nucleic acid molecule that should not be detected during the detection of the target nucleic acid molecule, regardless of homology with the sequence of the target nucleic acid molecule. As used herein, the term “non-target nucleic acid sequence” refers to a nucleic acid sequence of a non-target nucleic acid molecule.

According to an embodiment of the present invention, the non-target nucleic acid sequence data set for the non-target nucleic acid molecule may be provided including information of alignment with a group representative sequence. More specifically, the non-target nucleic acid sequence data set for the non-target nucleic acid molecule may be provided in the form of an alignment file in which a group representative sequence is aligned with the non-target nucleic acid sequence of the non-target nucleic acid sequence data set.

The non-target nucleic acid sequence data set for the non-target nucleic acid molecule provided by such a method contains sequences similar to the target nucleic acid molecule but also contains nucleic acid sequences other than target nucleic acid sequences. Therefore, in the designing or providing of an oligonucleotide for detecting a target nucleic acid molecule, the oligonucleotide is designed so as not to hybridize with the nucleic acid sequences included in the non-target nucleic acid sequence data set for the non-target nucleic acid molecule, thereby causing no a risk of false-positive and providing an oligonucleotide with high specificity, for detecting a target nucleic acid molecule.

According to an embodiment of the present invention, the non-target nucleic acid sequence data set provided in step (g) satisfies at least one of the following homology criteria:
(i) the non-target nucleic acid sequence data set needs to have a homology of a predetermined value or more with a partial sequence region of at least one representative sequence of the group representative sequence and the taxonomic representative sequence;
(ii) the non-target nucleic acid sequence data set needs to have a homology of a predetermined value or more with at least one representative sequence of the group representative sequence and the taxonomic representative sequence; and
(iii) the non-target nucleic acid sequence data set having homology criterion (i) needs to have homology criterion (ii).

According to the present invention, in step (e), nucleic acid sequence data having a homology of at least a predetermined value with the group representative sequence are retrieved; and in step (g), nucleic acid sequence data not associated with the organism received in step (a) are provided among the retrieved nucleic acid sequence data and then nucleic acid sequence data having a homology of at least a predetermined value with at least one representative sequence of the group representative sequence and the taxonomic representative sequence in the nucleic acid sequence data can be provided as a non-target nucleic acid sequence data set. Therefore, the present embodiment may also be expressed by the above-described time-series process.

According to homology criterion (i) in the present embodiment, the non-target nucleic acid sequence data set should have a homology of a predetermined value or more with a partial sequence region of at least one representative sequence of the group representative sequence and the taxonomic representative sequence.

The partial sequence region of at least one representative sequence of the group representative sequence and the taxonomic representative sequence means a region having a predetermined nucleotide length from one end of the non-target nucleic acid sequence included in the non-target nucleic acid sequence data set and the at least one representative sequence, which is aligned with the at least one representative sequence for homology comparison, and specifically, the nucleotide length of the partial sequence region is 10 bp, 20 bp, 30 bp, 40 bp, 50 bp, 60 bp, or 70 bp, but is not limited thereto.

As for the at least a predetermined value of the homology of the partial sequence region, the at least a predetermined value of the homology is preferably larger considering the design requirements wherein an oligonucleotide needs to be designed so as not to detect a non-target nucleic acid sequence having high homology in the partial sequence region. Specifically, the at least a predetermined value is at least 80%, at least 90%, at least 95%, at least 98%, or at least 99%, or 100%.

According to homology criterion (ii) in the present embodiment, the non-target nucleic acid sequence data set should have a homology of at least a predetermined value with at least one representative sequence of the group representative sequence and the taxonomic representative sequence.

According to homology criterion (ii) in the present embodiment, the non-target nucleic acid sequence data set should have a homology of at least a predetermined value with at least one representative sequence.

Since homology criterion (ii) is compared with the at least one representative sequence, the predetermined value of the homology may be lower than homology criterion (i). Specifically, the predetermined value of homology criterion (ii) is at least 60%, at least 70%, at least 80%, at least 90%, or at least 95%, or 100%.

According to homology criterion (iii) of the present embodiment, both of homology criteria (i) and (ii) need to be required.

In the present embodiment, unlike in step (e), in the nucleic acid sequence data not associated with the organism of interest from the nucleic acid sequence data set having homology with the group representative sequence, a nucleic acid sequence data set having homology with the group representative sequence and/or the taxonomic representative sequence is additionally provided as a non-target nucleic acid sequence data set.

The reason for considering not only the group representative sequence but also the taxonomic representative sequence in the present embodiment is to retrieve the non-target nucleic acid sequence data that should not be detected by the oligonucleotide, without omission, in the design of an oligonucleotide used to detect the target nucleic acid molecule.

In the present embodiment, the reason for requiring the homology criteria of non-target nucleic acid sequences is that false-positive errors due to the non-target nucleic acid molecule similar to the target nucleic acid molecule become more problematic when a part of the non-target nucleic acid molecule shows very high similarity to the sequence of the target nucleic acid molecule.

In general, only a specific region has very high homology with the target nucleic acid sequence but the homology of the other region is low, and resultantly, a non-target nucleic acid sequence entirely having low sequence homology with the target nucleic acid molecule may not be considered in the design of an oligonucleotide for detecting a target nucleic acid molecule. In order to solve such a problem, in the method of the present invention, non-target sequences, of which the entirety has somewhat low homology with the target nucleic acid molecule but a partial region has high homology are separately selected, and information thereabout is provided.

### Steps (h) and (j): Classifying as Design Exclusion Target Nucleic Acid Sequence Data

According to an embodiment of the present invention, the method further includes the following steps:
(h) retrieving nucleic acid sequence data having a homology of a predetermined value or more with the group representative sequence to provide a nucleic acid sequence data set; and
(j) classifying, as design exclusion target nucleic acid sequence data, target nucleic acid sequence data of the group representative sequence and target nucleic acid sequence data belonging to the same group as the group representative sequence in the target nucleic acid sequence data set in step (f), when the taxonomic name and/or taxonomic ID of the organism associated with the group representative sequence satisfies one of the following predetermined criteria, wherein the predetermined criteria include the following:
(i) nucleic acid sequence data corresponding to the taxonomic name and/or taxonomic ID of the organism associated with the group representative sequence are absent and only nucleic acid sequence data corresponding to a taxonomic name and/or taxonomic ID of an organism different from the organism associated with the group representative sequence are present in the nucleic acid sequence data set provided in step (h);
(ii) the homology of nucleic acid sequence data corresponding to the taxonomic name and/or taxonomic ID of the organism associated with the group representative sequence is lower than the homology of nucleic acid sequence data corresponding to a taxonomic name and/or taxonomic ID of an organism different from the organism associated with the group representative sequence in the nucleic acid sequence data set provided in step (h);
(iii) nucleic acid sequence data corresponding to the taxonomic name and/or taxonomic ID of the organism associated with the group representative sequence are absent in the nucleic acid sequence data set provided in step (h), and the proportion of nucleic acid sequence data of an organism corresponding to a superclass taxonomic name and/or taxonomic ID of the organism associated with the group representative sequence or a subclass taxonomic name and/or taxonomic ID of the superclass is less than a predetermined value relative to the nucleic acid sequence data set provided in step (h); and
(iv) all of the nucleic acid sequence data set provided in step (h) is associated with a nucleic acid sequence data set of the organism associated with the group representative sequence, but a name of a target nucleic acid molecule described in a descriptor of each of nucleic acid records containing the nucleic acid sequence data set is absent or different from at least one of the name of the target nucleic acid molecule and the retrieved synonyms.

According to the present invention, the target nucleic acid sequence data set for the target nucleic acid molecule is a nucleic acid sequence data set wherein in the design of an oligonucleotide used to amplify or detect the target nucleic acid molecule of the organism of interest, the oligonucleotide needs to be considered so as to necessarily hybridize with the target nucleic acid sequence data set.

However, the design of the oligonucleotide to hybridize with all the target nucleic acid sequence data set may have a problem as below. If there is a registration error in the nucleotide database in the retrieved target nucleic acid sequence data (the same organism as the organism of interest has been registered in the database but the registered nucleic acid sequence is a nucleic acid sequence of a different organism from the organism of interest), the design of an oligonucleotide so as to hybridize with all the nucleic acid sequences with such a registration error not only lowers the target-coverage of the oligonucleotide, but also introduces degenerate bases into the oligonucleotide or increases the number of combinations thereof in order to increase the target-coverage.

Therefore, as in the present embodiment, the registration error of the group representative sequence is identified, and target nucleic acid sequence data excluded from the design of the oligonucleotide need to be classified from the target nucleic acid sequence data set for the target nucleic acid molecule.

According to an embodiment of the present invention, the nucleic acid sequence data having the homology in step (h) are retrieved from a third database.

The third database used to retrieve the nucleic acid sequence data having the homology in step (h) of the present embodiment may be identical to the above-described second database or may be a nucleotide database including a partial nucleotide database of the second database.

When the third database includes a partial nucleotide database of the second database, the third database may be a nucleotide database including NCBI GenBank (including SNP and non-WGS databases), RefSeq, DDBJ, and EMBL databases, or a nucleotide database constructed by downloading the nucleotide database.

In the present embodiment, it has been expressed in step (h) that nucleic acid sequence data having a homology of at least a predetermined value with the group representative sequence are retrieved (specifically, from the third database), but the nucleic acid sequence data retrieved and provided (specifically, from the second database) in step (e) may be used as the nucleic acid sequence data set provided in step (h). Specifically, when the third database and the second database are identical to each other, with respect to the nucleic acid sequence data satisfying the homology criteria of step (h) among the nucleic acid sequence data retrieved in step (e), it is checked whether the taxonomic name and/or taxonomic ID of the organism associated with the group representative sequence satisfies one of the predetermined criteria above.

Alternatively, when the third database is included in a part of the nucleotide database of the second database, nucleic acid sequence data corresponding to the third database are retrieved from the nucleic acid sequence data retrieved in step (e), and with respect to the nucleic acid sequence data satisfying the homology criteria of step (h) among the retrieved nucleic acid sequence data, it is checked whether the taxonomic name and/or taxonomic ID of the organism associated with the group representative sequence satisfies one of the predetermined criteria above.

In step (j) of the present embodiment, when the retrieval results of the selected group representative sequence and the nucleic acid sequence data having homology therewith are compared with each other, and the taxonomic name and/or taxonomic ID of the organism associated with the group representative sequence satisfies one of the predetermined criteria, the target nucleic acid sequence data of the group representative sequence and the target nucleic acid sequence data belonging to the same group as the group representative sequence in the target nucleic acid sequence data set in step (f) are classified as design exclusion target nucleic acid sequence data.

When some of the target nucleic acid sequence data is classified as design exclusion target nucleic acid sequence data, an oligonucleotide is not designed with reference to the design exclusion target nucleic acid sequence data in the design of the oligonucleotide. Therefore, the oligonucleotide is designed based on the target nucleic acid sequence data from which the design exclusion target nucleic acid sequence data are excluded. That is, the design exclusion target nucleic acid sequence data are nucleic acid sequences that may or may not be detected by the oligonucleotide.

The predetermined value of the homology considered in step (h) may be larger than the homology considered in step (e), but may be equal to the homology reference of the target nucleic acid sequence data set in step (f).

The predetermined value or more of the homology in step (h) may be a homology of 70% or more, 80% or more, 90% or more, 95% or more, 96% or more, 97% or more, 98% or more, or 99% or more, or 100%. The predetermined value may be a value in a predetermined range, and may be 70% to 100%, 80% to 100%, 90% to 100%, or 95% to 100%, but is not limited thereto.

Less than the predetermined value in the proportion of nucleic acid sequence data in criterion (iii) in step (j) may be less than 2%, less than 5%, less than 8%, less than 10%, less than 15%, less than 20%, less than 25%, less than 30%, less than 35%, or less than 40%.

Among remaining target nucleic acid sequence data except for the design exclusion nucleic acid sequence data from the target nucleic acid sequence data, there may be a plurality of target nucleic acid sequence data having the same accession number in one group or in multiple groups. In such a case, nucleic acid sequence data that overlap partially or entirely in the plurality of target nucleic acid sequence data may be merged and provided.

According to an embodiment of the present invention, the method may further include, after step (j), step (j-1) of, when there are a plurality of nucleic acid sequence data having the same accession number in multiple groups among remaining target nucleic acid sequence data except for the design exclusion nucleic acid sequence data from the target nucleic acid sequence data provided in step (f), merging target nucleic acid sequence data that overlap partially or entirely among the plurality of target nucleic acid sequence data and providing the target nucleic acid data.

For example, even target nucleic acid sequence data having the same accession number may be present in multiple groups due to a difference of sites having homology with multiple group representative sequences, respectively, and hence, the target nucleic acid sequence data set may comprise redundant sequences. In such a case, it is not appropriate to use each of the plurality of nucleic acid sequence data as the target nucleic acid sequence of the target nucleic acid molecule, and it is preferable to handle, as a target nucleic acid sequence for the target nucleic acid molecule, one nucleic acid sequence obtained by merging the plurality of nucleic acid sequence data.

According to an embodiment of the present invention, the method further includes, after step (j-1), step (j-2) of comparing the merged target nucleic acid sequence data with the group representative sequences in the multiple groups in view of homology and allowing the merged target nucleic acid sequence data to be included in a group to which a group representative sequence with higher homology belongs.

In the present embodiment, a plurality of target nucleic acid sequence data included in multiple groups are merged and then a group for one target nucleic acid sequence data resulting from merging is determined.

Through such a method, one nucleic acid sequence data obtained by merging nucleic acid sequence data overlapping partially or entirely are provided, thereby reducing a probability of statistical errors in the analysis based on a target nucleic acid sequence data set, which occur when target nucleic acid sequence data of a part of the target nucleic acid molecule are recognized as target nucleic acid sequence data for each independent target nucleic acid molecule.

### Steps (k) and (l): Classifying as Design Exclusion Non-Target Nucleic Acid Sequence Data

According to an embodiment of the present invention, the method further includes the following steps:
(k) retrieving nucleic acid sequence data having a homology of a predetermined value or more with the non-target nucleic acid sequence of the organism associated with the non-target nucleic acid sequence data set to provide a nucleic acid sequence data set; and
(l) classifying, as design exclusion non-target nucleic acid sequence data, non-target nucleic acid sequence data of the organism in the non-target nucleic acid sequence data set in step (k), when the taxonomic name and/or taxonomic ID of the organism associated with the non-target nucleic acid sequence satisfies one criterion of the following predetermined criteria, wherein the predetermined criteria include the following:
(i) nucleic acid sequence data corresponding to the taxonomic name and/or taxonomic ID of the organism associated with the non-target nucleic acid sequence are absent and only nucleic acid sequence data corresponding to a taxonomic name and/or taxonomic ID of an organism different from the organism are present in the nucleic acid sequence data set provided in step (k);
(ii) the homology of the non-target nucleic acid sequence data corresponding to the taxonomic name and/or taxonomic ID of the organism associated with the non-target nucleic acid sequence is lower than the homology of nucleic acid data corresponding to a taxonomic name and/or taxonomic ID of an organism different from the organism in the nucleic acid sequence data set provided in step (k); and
(iii) non-target nucleic acid sequence data corresponding to the taxonomic name and/or taxonomic ID of the organism associated with the non-target nucleic acid sequence are absent in the nucleic acid sequence data set provided in step (k), and the proportion of nucleic acid sequence data of an organism corresponding to a superclass taxonomic name and/or taxonomic ID of the organism or a subclass taxonomic name and/or taxonomic ID of the superclass is less than a predetermined value relative to the nucleic acid sequence data set provided in step (k).

According to the present invention, the non-target nucleic acid sequence data set for the non-target nucleic acid molecule is a nucleic acid sequence data set wherein in the design of an oligonucleotide used to amplify or detect the target nucleic acid molecule of the organism of interest, the oligonucleotide needs to be considered so as not to necessarily hybridize with the non-target nucleic acid sequence data set.

However, the design of the oligonucleotide not to hybridize with all the target nucleic acid sequence data set may have a problem as below. There may be a registration error of the retrieved non-target nucleic acid data in the nucleotide database (e.g., a non-target nucleic acid sequence has been registered as organism A in a database but the registered nucleic acid sequence is a nucleic acid sequence of an organism different from organism A), or due to a high homology with the group representative sequence, it may be often difficult to design an oligonucleotide so as not to detect the retrieved non-target nucleic acid sequence.

Therefore, as in this embodiment, it is necessary to determine the design exclusion non-target nucleic acid sequence data set, which are not considered in the design of the oligonucleotide in the non-target nucleic acid sequence data set by checking the registration error or the like of the organism for the retrieved non-target nucleic acid sequence data.

According to an embodiment of the present invention, the nucleic acid sequence data having the homology in step (k) are retrieved from a third database.

The third database used to retrieve the nucleic acid sequence data having the homology in step (k) of the present embodiment may be identical to the above-described second database or may be a nucleotide database including a partial nucleotide database of the second database.

When the third database includes a partial nucleotide database of the second database, the third database may be a nucleotide database including NCBI GenBank (including SNP and non-WGS databases), RefSeq, DDBJ, and EMBL databases, or a nucleotide database constructed by downloading the nucleotide database.

According to an embodiment of the present invention, the non-target nucleic acid sequence data in step (k) is a non-target nucleic acid sequence data set having a homology of at least a predetermined value with the group representative sequence in the non-target nucleic acid sequence data set provided in step (g), and the predetermined value of the homology may be identical to the predetermined value of the homology of the target nucleic acid sequence data set provided in step (f). Specifically, the at least a predetermined value in the homology may be a homology of at least 70%, at least 80%, at least 90%, at least 95%, at least 96%, at least 97%, at least 98%, or at least 99%, or 100%. The predetermined value may be a value in a predetermined range, and may be 70% to 100%, 80% to 100%, 90% to 100%, or 95% to 100%, but is not limited thereto.

The predetermined value of the homology of the nucleic acid sequence data retrieved in step (k) may be a homology of at least 70%, at least 80%, at least 90%, at least 95%, at least 96%, at least 97%, at least 98%, or at least 99%, or 100%. The predetermined value may be a value in a predetermined range, and may be 70% to 100% to 100%, 90% to 100%, or 95% to 100%, but is not limited thereto.

In step (l) of the present embodiment, when the retrieval results of the non-target nucleic acid sequence of the organism for the non-target nucleic acid sequence data set and the nucleic acid sequence data having homology therewith are compared with each other, and the taxonomic name and/or taxonomic ID of the organism for the non-target nucleic acid sequence satisfies one of the predetermined criteria, the non-target nucleic acid sequence data of the organism in the non-target nucleic acid sequence data set in step (g) are classified as design exclusion non-target nucleic acid sequence data.

When some of the non-target nucleic acid sequence data is classified as design exclusion non-target nucleic acid sequence data, an oligonucleotide is not designed with reference to the design exclusion non-target nucleic acid sequence data in the design of the oligonucleotide. Therefore, the oligonucleotide is designed so as not to hybridize with remaining non-target nucleic acid sequence data except for the design exclusion non-target nucleic acid sequence data from the non-target nucleic acid sequence data. That is, the design exclusion non-target nucleic acid sequence data are nucleic acid sequences that may or may not be detected by the oligonucleotide.

Less than the predetermined value in the proportion of nucleic acid sequence data in criterion (iii) in step (l) may be less than 2%, less than 5%, less than 8%, less than 10%, less than 15%, less than 20%, less than 25%, less than 30%, less than 35%, or less than 40%.

### Storage Medium, Device, and Program

In another aspect of the present invention, there is provided a computer readable storage medium containing instructions to configure a processor to perform a method for providing a nucleic acid sequence data set for the design of an oligonucleotide used to detect a target nucleic acid molecule of an organism of interest, the method including: (a) receiving a name of the target nucleic acid molecule and a name of the organism of interest and retrieving synonyms for the target nucleic acid molecule of the organism of interest; (b) retrieving nucleic acid sequence data included in nucleic acid records, wherein each of the nucleic acid records is associated with the organism of interest and comprises a descriptor in which at least one of the name of the target nucleic acid molecule and the retrieved synonyms are described; (c) sorting the retrieved nucleic acid data according to taxonomic name and/or taxonomic identification (ID) and selecting taxonomic representative sequences among nucleic acid sequence data having the same taxonomic name and/or taxonomic ID; (d) grouping the selected taxonomic representative sequences according to homology and selecting a group representative sequence for each group; and (e) retrieving nucleic acid sequence data having a homology of at least a predetermined value with the group representative sequence to provide the retrieved nucleic acid sequence data as a nucleic acid sequence data set for the design of an oligonucleotide.

In still another aspect of the present invention, there is provided a computer program to be stored on a computer readable storage medium, to configure a processor to perform a method for providing a nucleic acid sequence data set for the design of an oligonucleotide used to detect a target nucleic acid molecule of an organism of interest, the method including: (a) receiving a name of the target nucleic acid molecule and a name of the organism of interest and retrieving synonyms for the target nucleic acid molecule of the organism of interest; (b) retrieving nucleic acid sequence data included in nucleic acid records, wherein each of the nucleic acid records is associated with the organism of interest and comprises a descriptor in which at least one of the name of the target nucleic acid molecule and the retrieved synonyms are described; (c) sorting the retrieved nucleic acid data according to taxonomic name and/or taxonomic identification (ID) and selecting taxonomic representative sequences among nucleic acid sequence data having the same taxonomic name and/or taxonomic ID; (d) grouping the selected taxonomic representative sequences according to homology and selecting a group representative sequence for each group; and (e) retrieving nucleic acid sequence data having a homology of at least a predetermined value with the group representative sequence to provide the retrieved nucleic acid sequence data as a nucleic acid sequence data set for the design of an oligonucleotide.

In another aspect of the present invention, there is provided a device for providing a nucleic acid sequence data set for the design of an oligonucleotide used to detect a target nucleic acid molecule of an organism of interest, the device including (a) a computer processor and (b) a computer readable storage medium of the present invention coupled to the computer processor.

Since the storage medium, the device, and the computer program of the prevent invention are intended to perform the present methods described as above in a computer, the common descriptions among them are omitted in order to avoid undue redundancy leading to the complexity of this specification.

The program instructions are operative, when performed by the processor, to cause the processor to perform the present method described above. The program instructions for performing the method of providing a nucleic acid sequence data set for the design of an oligonucleotide used to detect a target nucleic acid molecule of an organism of interest may include the following instructions: (i) an instruction to receive a name of the target nucleic acid molecule and a name of the organism of interest and retrieve synonyms for the target nucleic acid molecule of the organism of interest; (ii) an instruction to retrieve nucleic acid sequence data included in nucleic acid records, wherein each of the nucleic acid records is associated with the organism of interest and comprises a descriptor in which at least one of the name of the target nucleic acid molecule and the retrieved synonyms are described; (iii) an instruction to sort the retrieved nucleic acid data according to taxonomic name and/or taxonomic identification (ID) and select taxonomic representative sequences among nucleic acid sequence data having the same taxonomic name and/or taxonomic ID; (iv) an instruction to group the selected taxonomic representative sequences according to homology and select a group representative sequence for each group; and (v) an instruction to retrieve nucleic acid sequence data having a homology of a predetermined value or more with the group representative sequence to provide (e.g., to display on an output device) the retrieved nucleic acid sequence data as a nucleic acid sequence data set for the design of an oligonucleotide.

The method of the present invention is implemented in a processor, wherein the processor may be a processor in a stand-alone computer, a network attached computer, or a data acquisition device, such as a real-time PCR device.

The types of the computer readable storage medium include various storage media, for example, CD-R, CD-ROM, DVD, flash memory, floppy disk, hard drive, portable HDD, USB, magnetic tape, MINIDISC, nonvolatile memory card, EEPROM, optical disk, optical storage medium, RAM, ROM, system memory and web server, but are not limited thereto.

The nucleic acid sequence data set for the design of an oligonucleotide used to detect a target nucleic acid molecule of an organism of interest may be provided in various manners. For example, the nucleic acid sequence data set for the design of an oligonucleotide may be provided to a separate system, such as a desktop computer system, via a network connection (e.g., LAN, VPN, intranet, and internet) or a direct connection (e.g., USB or other direct wired or wireless connection), or may be provided on a portable medium such as a CD, DVD, floppy disk and portable HDD. Similarly, the nucleic acid sequence data set for the design of an oligonucleotide may be provided to a server system via a network connection (e.g., LAN, VPN, Internet, intranet and wireless communication network) to a client, such as a notebook or a desktop computer system.

The instructions to configure the processor to perform the present invention may be included in a logic system. The instructions may be downloaded and stored in a memory module (e.g., hard drive or other memory, such as a local or attached RAM or ROM), although the instructions can be provided on any software storage medium (e.g., portable HDD, USB, floppy disk, CD and DVD). A computer code for implementing the present invention may be implemented in a variety of coding languages, such as C, C++, Java, Visual Basic, VBScript, JavaScript, Perl and XML. In addition, a variety of languages and protocols may be used in external and internal storage and transmission of data and commands according to the present invention.

The computer processor may be constructed in such a manner that a single processor can make several performances. Alternatively, the processor unit may be constructed in such a manner that several processors make the several performances, respectively.

The features and advantages of the present invention are summarized as follows:

(a) According to the conventional method of providing a nucleic acid sequence data set for the design of an oligonucleotide used to detect a target nucleic acid molecule of an organism of interest, nucleic acid sequence data were retrieved using a keyword, such as a name of the target nucleic acid molecule; the retrieved nucleic acid sequence data were sorted according to the sequence length to determine the longest sequence as a representative sequence; nucleic acid data having a homology of a predetermined value or more with the representative sequence were grouped; and then the nucleic acid data retrieved by using the keyword and the nucleic acid sequence data having homology with the representative sequence were incorporated, and provided as a target nucleic acid sequence data set for the target nucleic acid molecule. In addition, for use in the design of an oligonucleotide, an alignment file for the target nucleic acid sequence data was provided.

As a result, the alignment was not properly formed due to a difference in homology between the sequences retrieved by the representative sequences, and this required unnecessary time for analysts to review aligned nucleic acid sequences.

(b) In order to solve the above-described problems, in the present invention, nucleic acid sequence data retrieved by a synonym for a target nucleic acid molecule are sorted according to the taxonomic name and/or taxonomic ID; taxonomic representative sequences are selected among nucleic acid sequence data having the same taxonomic name and/or taxonomic ID; and the selected taxonomic representative sequences are grouped according to the homology to select a group representative sequence in each group; and then nucleic acid sequence data having a homology of a predetermined value or more with the group representative sequence are provided.

As a result, it was confirmed that the plurality of target nucleic acid sequences for the target nucleic acid molecule were retrieved without omission and the alignment results of the retrieved multiple target nucleic acid sequences were properly formed so as to be referred to in the design of an oligonucleotide.

(c) According to the present invention, the alignment results of the nucleic acid sequence data set are properly formed so as to be used to design an oligonucleotide, and thus the problems of time consumption and labor consumption resulting from analyst's reviewing of registration errors of sequences included in the retrieved nucleic acid sequence data set have been solved.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Example 1: Providing Nucleic Acid Sequence Data Set for Design of Oligonucleotide Used to Detect sopB Gene of Salmonella enterica

A program (AutoMSA v3.0) for providing a nucleic acid sequence data set for the design of an oligonucleotide used to detect a target nucleic acid molecule of an organism of interest was run to provide a nucleic acid sequence data set for the design of an oligonucleotide used to detect sopB gene of Salmonella enterica.

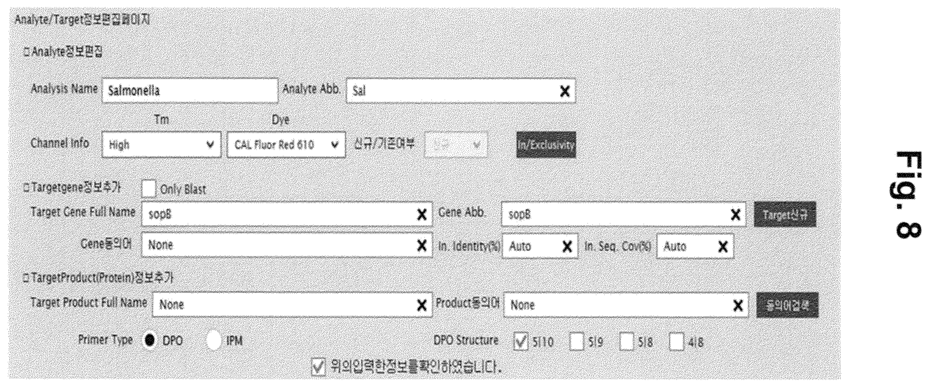

The scientific name of Salmonella enterica and the gene name (sopB) thereof were input in the user interface (UI) window of the AutoMSA v3.0 program (FIG. 8), and the AutoMSA v3.0 program was run.

The AutoMSA v3.0 program was run to proceed in the following order: (1) The gene name (sopB) and Salmonella enterica were received, and synonyms of sopB of Salmonella enterica (protein name: inositol phosphatase, inositol phosphate phosphatase, and the like) were retrieved from a gene database (a gene database constructed by downloading the gene database of NCBI being used). Specifically, when sopB is described in the gene symbol on the Full Report of a gene information summary record, inositol phosphatase, inositol phosphate phosphatase, or the like described in the gene description was retrieved as a synonym. In addition, inositol phosphatase, inositol phosphate phosphatase, or the like described in the Summary of the gene information summary record was retrieved as a synonym.

Then, Salmonella enterica, the gene name (sopB), and the synonym (protein name) of sopB were received, and nucleic acid sequence data included in nucleic acid records, wherein each of the nucleic acid records was associated with Salmonella enterica and included a descriptor in which the sopB and/or the synonym was described, were retrieved.

(2) Salmonella enterica, the gene name (sopB), and the synonym (protein name) of sopB were received, and identifiers of nucleic acid records, wherein each of the nucleic acid records was associated with Salmonella enterica and included a descriptor in which the sopB and/or the synonym was described, were retrieved from a nucleotide database (a nucleotide database constructed by downloading a nucleotide database including NCBI's nucleotide database, that is, NCBI GenBank (including STS, EST, GSS, SNP, TSA, PAT, WGS, and non-WGS), RefSeq, DDBJ, and EMBL databases being used). Specifically, the received Salmonella enterica, sopB, inositol phosphatase or inositol phosphate phosphatase, or the like was input as the query of the nucleotide database, and identifiers (specifically, accession number, GI, or the like) of nucleic acid records in which the organism name (taxonomic name), taxonomic ID, sopB, inositol phosphatase or inositol phosphate phosphatase, or the like was described in the title, gene, CDS, /gene, /product, /note, /taxon, or the like as the descriptor of the nucleic acid record was retrieved. (3) The nucleic acid sequence data specified by the identifiers were retrieved. Specifically, the nucleic acid records specified by the identifiers were retrieved, and nucleic acid sequence data were retrieved from nucleic acid records in which sopB, inositol phosphatase, or inositol phosphate phosphatase was identical to or included in the gene, CDS, /gene, /product, or /note as a descriptor of the nucleic acid records.

(4) The retrieved nucleic acid data were sorted according to the taxonomic name and/or taxonomic ID, and taxonomic representative sequences were selected among nucleic acid sequence data having the same taxonomic name and/or taxonomic ID. Specifically, the selecting of the taxonomic representative sequences from the nucleic acid sequence data having the same taxonomic name and/or taxonomic ID was performed as follows. First, the nucleic acid sequence data having the same taxonomic names and/or taxonomic IDs were sorted according to the sort criteria having the following order:
(i) sorting according to the assembly level of the nucleic acid sequence included in the nucleic acid sequence data, wherein as for the assembly level, complete genome, chromosome, scaffold, and config are ranked higher in that order (that is, the ranking of the complete genome is highest);
(ii) sorting according to whether the nucleic acid sequence data are included in a reference sequence (RefSeq) database, wherein the ranking is higher when the nucleic acid sequence data are included in the RefSeq database than when not included;
(iii) sorting according to whether a name of a nucleic acid molecule described in a descriptor of a nucleic acid record comprising the nucleic acid sequence data is identical to at least one of the received name of the target nucleic acid and the retrieved synonyms, wherein the ranking is higher when identical than when not identical;
(iv) sorting according to the length of the nucleic acid sequence data, wherein the longer the length, the higher the ranking;
(v) sorting according to whether there is description in a host of a descriptor of a nucleic acid record containing the nucleic acid sequence data, wherein the ranking is higher when a host of interest for the organism of interest is described in the host than when not described, and higher when an organism different from the organism of interest are not described in the host than when described;
(vi) sorting according to the registration date or revision date of a nucleic acid record comprising the nucleic acid sequence data, wherein the more recent the registration date or revision date, the higher the ranking; and
(vii) sorting according to the alphabetical order in accession number of the nucleic acid sequence data, wherein the earlier the alphabetical order of the accession number, the higher the ranking.

Then, the nucleic acid sequences of the highest-ranked nucleic acid sequence data among the sorted nucleic acid sequence data were selected as a taxonomic representative sequence. In sort criterion (v), the host of interest was Homo sapiens.

This procedure was performed on the nucleic acid sequence data sorted by taxonomic names and/or taxonomic IDs.

(5) The selected taxonomic representative sequences were grouped according to homology, and a group representative sequence was selected for each group. Specifically, the selected taxonomic representative sequences were sorted according to sort criteria having the following order:
(i) sorting according to the assembly level of the selected taxonomic representative sequence, wherein as for the assembly level, complete genome, chromosome, scaffold, and config are ranked higher in that order (that is, the ranking of the complete genome is highest);
(ii) the number of nucleic acid sequence data having the same taxonomic name and/or taxonomic ID as the selected taxonomic representative sequence, wherein the larger the number, the higher the ranking;
(iii) sorting according to whether there is a description in a host of a descriptor of a nucleic acid record containing the selected taxonomic representative sequence, wherein the ranking is higher when a host of interest for the organism of interest is described in the host than when not described, and higher when an organism different from the host of interest is not described in the host than when described; and
(iv) sorting according to the alphabetic order of accession number in the selected taxonomic representative sequence, wherein the earlier the alphabet order of the accession number, the higher the ranking. In sort criterion (iii), the host of interest was Homo sapiens.

The highest-ranked taxonomic representative sequence was selected among the sorted taxonomic representative sequences, and taxonomic representative sequences having a homology of 90% or more with the highest-ranked representative sequence were grouped using UCLUST algorithm, and the highest-ranked taxonomic representative sequence was selected as a group representative sequence for each group.

(6) By performing BLAST, nucleic acid sequence data having a homology of at least 50% (specifically, Identity: at least 50%, word size: 15, and E-value: 10000) with the group representative sequence were retrieved from a nucleotide database (specifically, the nucleotide database in (2) is used) and provided as the nucleic acid sequence data set for designing oligonucleotides. (7) The nucleic acid sequence data associated with the received Salmonella enterica in the provided nucleic acid sequence data were provided as a target nucleic acid sequence data set for the sopB gene. The provided target nucleic acid sequence data set had a homology of at least 90% (identity of 90% or more) with at least one representative sequence of the group representative sequence and the taxonomic representative sequence for the target nucleic acid sequence data set. The target nucleic acid sequence data set having a homology of at least 90% was provided as follows. Specifically, the sequences of the target nucleic acid sequence data set were extended to the length of the group representative sequence and/or taxonomic representative sequence, and then sequences having a query coverage of at least 10% and an identity of at least 90% on the basis of the length of the group representative sequence and/or taxonomic representative sequence were selected.

(8) The nucleic acid sequence data not associated with the received Salmonella enterica in the nucleic acid sequence data set provided in (6) were provided as a non-target nucleic acid sequence data set for a non-target nucleic acid molecule. The provided non-target nucleic acid sequence data set has (i) a homology of 100% (identify 100%) with a 20 bp sequence region of at least one representative sequence of the group representative sequence and the taxonomic representative sequence and (ii) a homology of at least 70% (identity of at least 70%) with at least one representative sequence of the group representative sequence and the taxonomic representative sequence.

Specifically, the sequences of the non-target nucleic acid sequence data set were extended to the length of the group representative sequence and/or taxonomic representative sequence, and while a 20 bp sequence region is shifted from one end of at least one representative sequence and/or each of the sequences of the non-target nucleic acid sequence data set, a non-target nucleic acid sequence data set having a homology of 100% (identity of 100%) with the 20 bp sequence region was selected, and (ii) sequences having a query coverage of 100% and an identity of at least 70% on the basis of the length of the group representative sequence and/or taxonomic representative sequence were selected from the selected non-target nucleic acid sequence data set.

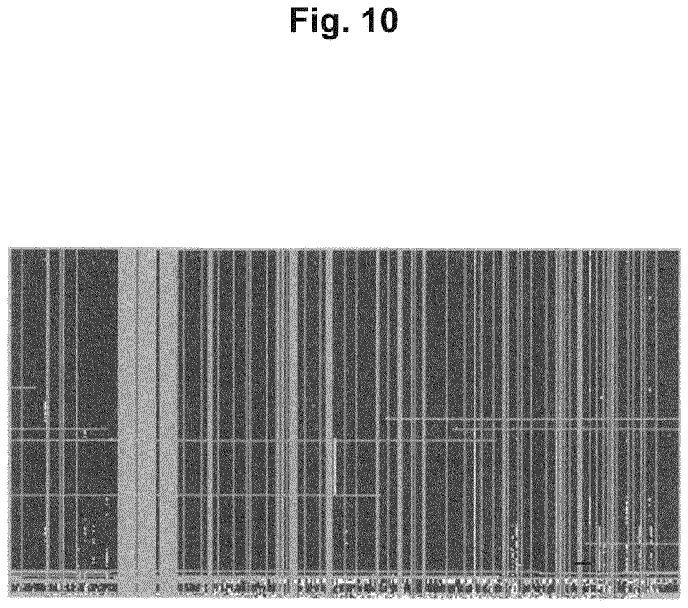

FIG. 10 shows the alignment results of the target nucleic acid sequence data set for the sopB gene of Salmonella enterica provided in (7). As can be confirmed from FIG. 10, as a result of aligning multiple target nucleic acid sequences, the alignment of the sequences was properly formed according to homology. It was determined from the alignment results in FIG. 10 that the more the black shades than gray shades, the alignment was more properly formed according to homology. The number of group representative sequences selected in (5) was 11.

As a results of running the AutoMSA v3.0 program according to Example 1, there were provided: a list of nucleic acid sequence data sets for the design of an oligonucleotide in (6); a list of target nucleic acid sequence data sets in (7); and a list of non-target nucleic acid sequence data sets in (8), each of which contains accession numbers, retrieved database information, nucleic acid sequence length, gene location information, the determination of whether it is a taxonomic representative sequence or a group representative sequence, organism names (taxonomic names), taxonomic IDs, homology information, biosample numbers, assembly levels, RefSeq number, and the like; and there were also provided: an alignment file of the target nucleic acid sequence data sets in (7); and an alignment file of the non-target nucleic acid sequence data sets in (8).

### Comparative Example 1: Providing Nucleic Acid Sequence Data Set for Design of Oligonucleotide Used to Detect Gene sopB of Salmonella enterica

A nucleic acid sequence data set for the design of an oligonucleotide used to detect sopB gene of Salmonella enterica in Comparative Example 1 was provided by AutoMSA program (AutoMSA v2.0) according to the method disclosed in PCT Publication No. WO2019/212238 previously applied by the present applicant.

The conventional AutoMSA program (AutoMSA v2.0) is same as Example 1 in view of program execution numbers (1) to (3), but differs from Example 1 in view of program execution numbers (4) to (8) as follows. The different portions from Example 1 will be described as follows.

The scientific name of Salmonella enterica and the gene name (sopB) were input in the user interface (UI) window of the AutoMSA v2.0 program (FIG. 8), and the AutoMSA v2.0 program was run.

The AutoMSA v2.0 program was run to proceed in the following order: (1) to (3) were equally performed as in Example 1. (4) The retrieved nucleic acid sequence data were sorted according to the sequence length; the longest nucleic acid sequence data were selected among the sorted nucleic acid sequence data; nucleic acid sequence data having a homology of at least 90% (identity of 90% or more) with the longest nucleic acid sequence data were grouped by using UCLUST algorithm; and the longest nucleic acid sequence data was selected as a group representative sequence in each group. (5) was equally performed by the same method as in (6) of Example 1. (6) was equally performed by the same method as in (7) of Example 1 except for the description with respect to the taxonomic representative sequence in (7) of Example 1, thereby providing a target nucleic acid sequence data set.

According to the conventional AutoMSA v2.0 program, the nucleic acid sequence data set retrieved in (3) and the nucleic acid sequence data set provided in (6) were incorporated, and provided as a target nucleic acid sequence data set for the design of an oligonucleotide.

The alignment results of the target nucleic acid sequence data set for the design of an oligonucleotide provided according to the conventional AutoMSA v2.0 program are shown in FIG. 2. As can be seen from FIG. 2, the alignment of the plurality of nucleic acid sequences was not properly formed according to homology considering many gray grades. As a result of examining the reason why the alignment was not properly formed as such, there were 25 group representative sequences, and the difference in sequence homology between the group representative sequences also caused a difference in homology between the sequences retrieved by group representative sequences, and thus the alignment of the multiple nucleic acid sequences was not properly formed.

### Example 2: Providing Nucleic Acid Sequence Data Set for Design of Oligonucleotide Used to Detect sopB Gene of Salmonella enterica

A method similar to that as in Example 1 was performed, but the AutoMSA v3.0 program, to which algorithms for automatically performing, on a program, the deletion of redundant sequences and the review of sequences having registration errors in the nucleotide database were further added, was run to provide a nucleic acid sequence data set for the design of an oligonucleotide used to detect the sopB gene of Salmonella enterica.

First, the protein name of the sopB gene was identified to be inositol phosphatase through the search from a gene database of NCBI.

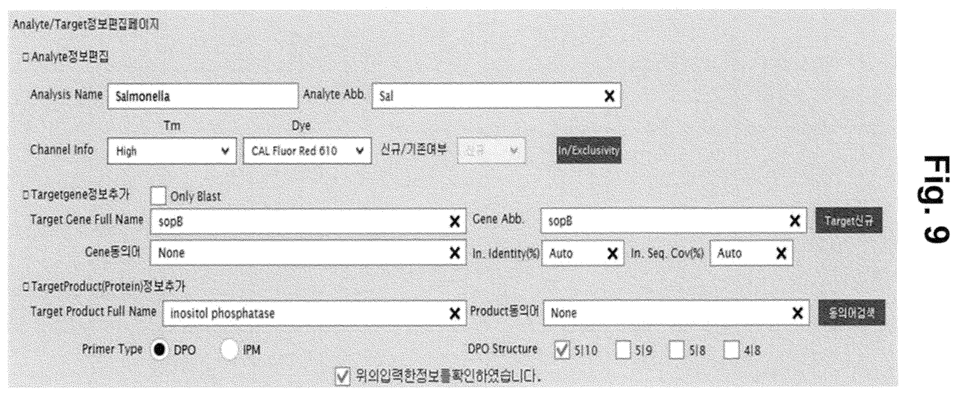

The scientific name of Salmonella enterica, gene name (sopB), and protein name (inositol phosphatase) were input in the user interface (UI) window of the AutoMSA v3.0 program (FIG. 9), and the AutoMSA v3.0 program was run.

The AutoMSA v3.0 program was run to proceed in the following order: (1) Salmonella enterica, the gene name (sopB), and the protein name (inositol phosphatase) were received, and a synonym of sopB of Salmonella enterica (protein name: inositol phosphate phosphatase, or the like) were retrieved from the gene database (identical to the gene database used in (1) of Example 1), and inositol phosphate phosphatase or the like, which is a protein name retrieved as a synonym of the sopB, was reviewed and then inositol phosphate phosphatase or the like was used as a synonym. Specifically, when sopB is described in the gene symbol on the Full Report of a gene information summary record, inositol phosphatase, inositol phosphate phosphatase, or the like described in the gene description was retrieved as a synonym. In addition, inositol phosphatase, inositol phosphate phosphatase, or the like described in the Summary of the gene information summary record was retrieved as a synonym. Since inositol phosphatase is a synonym inputted by a user before running the program, inositol phosphate phosphatase or the like is an additionally retrieved synonym.

Then, Salmonella enterica, the gene name (sopB), and the synonym (protein name) of sopB were received, and nucleic acid sequence data included in nucleic acid records, wherein each of the nucleic acid records was associated with Salmonella enterica and included a descriptor in which the sopB and/or the synonym was described, were retrieved.

(2) Salmonella enterica, the gene name (sopB), and the synonym (protein name) of sopB were received, and identifiers of nucleic acid records were retrieved from a nucleotide database, wherein each of the nucleic acid records is associated with Salmonella enterica and includes a descriptor in which sopB and/or the synonym (protein name) were described. Specifically, the received Salmonella enterica, sopB, inositol phosphatase, inositol phosphate phosphatase, or the like was inputted as the query of the nucleotide database (identical to the nucleotide database in (3) of Example 1), and identifiers (specifically, accession number, GI, and the like) of nucleic acid records in which the organism name (taxonomic name), taxonomic ID, sopB, inositol phosphatase, inositol phosphate phosphatase, or the like was descried in the title, gene, CDS, /gene, /product, /note, /taxon, or the like as the descriptor of the nucleic acid records were retrieved. (3) The nucleic acid sequence data specified by the identifiers were retrieved. Specifically, the nucleic acid records specified by the identifiers were retrieved, and nucleic acid sequence data were retrieved from nucleic acid records in which sopB was identical to or included in the gene, CDS, /gene as the descriptor of the nucleic acid records, and inositol phosphatase, or inositol phosphate phosphatase was identical to or included in the/product as the descriptor. When nucleic acid sequence data were not retrieved through the descriptor associated with the gene name and protein name, the nucleic acid sequence data were retrieved from nucleic acid records in which sopB, inositol phosphatase, or inositol phosphate phosphatase was identical to or included in the/note as the descriptor.

(3-1) Redundant sequences were deleted from the retrieved nucleic acid sequence data. Specifically, the retrieved nucleic acid sequence data were sorted according to biosample identification to select nucleic acid sequence data having the same biosample identification, and the selected nucleic acid sequence data were sorted according to sort criteria having the following order: (i) sorting the selected nucleic acid sequence data according to the assembly level, wherein as for the assembly level, complete genome, chromosome, scaffold, and config are ranked higher in that order (that is, the complete genome has the highest ranking); and (ii) sorting according to whether the selected nucleic acid sequence data are included in a reference sequence (RefSeq) database, wherein the ranking is higher when the nucleic acid sequence data are included in the RefSeq database than when not included.

The nucleic acid sequence of the highest-ranked nucleic acid sequence data was selected among the sorted nucleic acid sequence data, and the nucleic acid sequence data except for the highest-ranked nucleic acid sequence data were deleted from the retrieved nucleic acid sequence data.

(4 to 8) were equally performed by the same methods as in (4) to (8) of Example 1. However, sort criterion (iii) in (4) of Example 1 was replaced to perform with the following sort criterion: (iii) sorting according to whether at least one of the received name of the target nucleic acid molecule and the retrieved synonyms are identical to a name of a nucleic acid molecule and/or protein name described in a descriptor of a nucleic acid record containing the nucleic acid sequence data in the order of the nucleic acid molecule name and protein name, the protein name, and the nucleic acid molecule name, wherein the ranking is higher when identical in the above order (that is, the ranking is the highest when the at least one of the received name of the target nucleic acid molecule and the retrieved synonyms are identical to both the name of a nucleic acid molecule and protein name described in a descriptor of a nucleic acid record containing the nucleic acid sequence data) and the ranking is the lowest when no names are identical thereto.

(9) The registration errors were checked for the group representative sequence included in the target nucleic acid sequence data set provided in (7). Specifically, a method including the following steps was performed. The nucleic acid sequence data having a homology of at least 90% (identity of 90% or more) with the group representative sequence were retrieved from a nucleotide database (specifically, a nucleotide database constructed by downloading a nucleotide database including NCBI GenBank (including SNP and non-WGS databases), RefSeq, DDBJ, and EMBL databases is used) to provide a nucleic acid sequence data set (9-1) (alternatively, nucleic acid sequence data associated with Salmonella enterica were selected from the nucleic acid sequence data set in (6), nucleic acid sequence data having a homology of at least 90% with the group representative sequence data were selected, and then a nucleic acid sequence data set included in the nucleotide database in (9) was selected and provided). Then, when the taxonomic name and/or taxonomic ID of an organism associated with the group representative sequence satisfies one of the following predetermined criteria, target nucleic acid sequence data of the group representative sequence and target nucleic acid sequence data belonging to the same group as the group representative sequence in the target nucleic acid sequence data set in (7) were classified as design exclusion target nucleic acid sequence data. The predetermined criteria included the following: (i) nucleic acid sequence data corresponding to the taxonomic name and/or taxonomic ID of the organism associated with the group representative sequence are absent and only nucleic acid sequence data corresponding to a taxonomic name and/or taxonomic ID of an organism different from the organism associated with the group representative sequence are present in the nucleic acid sequence data set of (9-1);
(ii) the homology of nucleic acid sequence data corresponding to the taxonomic name and/or taxonomic ID of the organism associated with the group representative sequence is lower than the homology of target nucleic acid sequence data corresponding to a taxonomic name and/or taxonomic ID of an organism different from the organism associated with the group representative sequence, in the nucleic acid sequence data set of (9-1);
(iii) target nucleic acid sequence data corresponding to the taxonomic name and/or taxonomic ID of the organism associated with the group representative sequence are absent in the nucleic acid sequence data set of (9-1), and the proportion of nucleic acid sequence data of an organism corresponding to a superclass taxonomic name and/or taxonomic ID of the organism associated with the group representative sequence or a subclass taxonomic name and/or taxonomic ID of the superclass is less than 10% relative to the nucleic acid sequence data set of (9-1); and
(iv) all of the nucleic acid sequence data set of (9-1) is associated with a nucleic acid sequence data set of the organism associated with the group representative sequence, but a name of a target nucleic acid molecule described in the descriptors of nucleic acid records containing the nucleic acid sequence data set is absent or different from at least one of the name of the target nucleic acid molecule and the retrieved synonyms.

Since the group representative sequence satisfying the above predetermined criteria in (9) was a group representative sequence with a registration error, the group representative sequence with a registration error and target nucleic acid sequence data set belonging to the same group as the group representative sequence in the target nucleic acid sequence data set provided in (7) were classified as a design exclusion target nucleic acid sequence data set, which was not used in the design of an oligonucleotide.

(10) The deletion of redundant sequences in (3-1) was conducted after (6) or (9).

(11) It was checked whether a registration error was present in the non-target nucleic acid sequence data set provided in (8). Specifically, a method including the following steps was performed. The nucleic acid sequence data having a homology of at least 90% with a non-target nucleic acid sequence of the organism associated with the non-target nucleic acid sequence data set were retrieved from the nucleotide database (specifically, a nucleotide database constructed by downloading a nucleotide database including NCBI GenBank (including SNP and non-WGS databases), RefSeq, DDBJ, and EMBL databases is used) to provide a nucleic acid sequence data set (11-1). In addition, when the taxonomic name and/or taxonomic ID of the organism associated with the non-target nucleic acid sequence satisfies one of the following predetermined criteria, the non-target nucleic acid sequence data of the organism in the non-target nucleic acid sequence data set were classified as design exclusion non-target nucleic acid sequence data. The predetermined criteria included the following: (i) nucleic acid sequence data corresponding to the taxonomic name and/or taxonomic ID of the organism associated with the non-target nucleic acid sequence are absent and only nucleic acid sequence data corresponding to a taxonomic name and/or taxonomic ID of an organism different from the organism are present in the nucleic acid sequence data set of (11-1);
(ii) the homology of the non-target nucleic acid sequence data corresponding to the taxonomic name and/or taxonomic ID of the organism for the non-target nucleic acid sequence is lower than the homology of nucleic acid data corresponding to a taxonomic name and/or taxonomic ID of an organism different from the organism, in the nucleic acid sequence data set of (11-1); and
(iii) the non-target nucleic acid sequence data corresponding to the taxonomic name and/or taxonomic ID of the organism associated with the non-target nucleic acid sequence are absent in the nucleic acid sequence data set of (11-1), and the proportion of nucleic acid sequence data of an organism corresponding to a superclass taxonomic name and/or taxonomic ID of the organism or a subclass taxonomic name and/or taxonomic ID of the superclass is less than 10% relative to the nucleic acid sequence data set of (11-1).

Since the non-target nucleic acid sequence data set of the organism satisfying the above predetermined criteria in (11) is a non-target nucleic acid sequence data set with a registration error, the non-target nucleic acid sequence data for the organism with a registration error in the non-target nucleic acid sequence data set provided in (8) were classified as a design exclusion non-target nucleic acid sequence data set, which was not used in the design of an oligonucleotide.

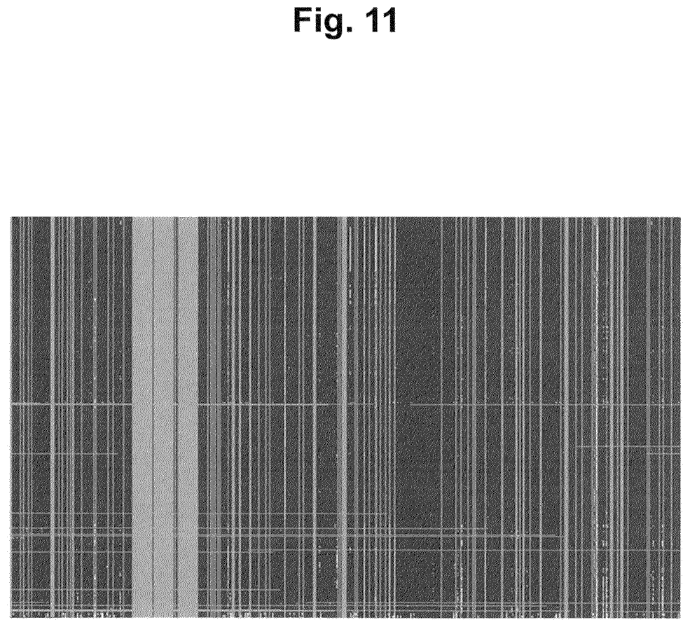

FIG. 11 shows the alignment results of the target nucleic acid sequence data set for the sopB gene of Salmonella enterica provided after (10). As can be confirmed from FIG. 11, as a result of aligning multiple target nucleic acid sequences, the alignment of the sequences was properly formed according to homology considering that black shades were more than gray shades. Five group representative sequences were selected in (10), and a total of 1,549 of taxonomic names or taxonomic IDs were included in the finally retrieved target nucleic acid sequence data set, and the number of target nucleic acid sequence data finally retrieved was 13,989.

As a result of comparing FIG. 10 showing the alignment results provided in Example 1 and FIG. 11 showing the alignment results provided in Example 2, it was confirmed that the alignment of multiple target nucleic acid sequences was more properly formed in FIG. 11.

As a results of running the AutoMSA v3.0 program according to Example 2, there were provided: a list of nucleic acid sequence data sets for the design of an oligonucleotide in (6); a list of target nucleic acid sequence data sets in (7); a list of non-target nucleic acid sequence data sets in (8); a list of nucleic acid sequence data deleted due to redundant sequences in (3-1) and (10); a list of design exclusion target nucleic acid sequence data sets classified in (9); and list of design exclusion non-target nucleic acid sequence data sets classified in (11), each of which contains information of accession numbers, retrieved database information, nucleic acid sequence length, gene location information, the determination of whether it is a taxonomic representative sequence or a group representative sequence, organism names (taxonomic names), taxonomic IDs, homology information, biosample numbers, assembly levels, RefSeq number, and the like. There were also provided: an alignment file of the target nucleic acid sequence data sets in (7); and an alignment file of the non-target nucleic acid sequence data sets in (8).

### Comparative Example 2: Providing Nucleic Acid Sequence Data Set for Design of Oligonucleotide Used to Detect sopB Gene of Salmonella enterica

The user who received the alignment file of FIG. 2 provided by the AutoMSA program (AutoMSA v2.0) in Comparative Example 1 could not design an oligonucleotide from the alignment result of FIG. 2, and therefore, reviewed a group representative sequence having a registration error among the aligned nucleic acid sequences to delete a group representative sequence breaking the alignment between the plurality of nucleic acid sequences. If the user conducts a review process, such as deleting a group representative sequence, nucleic acid sequence data having homology with a changed group representative sequence need to be again retrieved from the nucleotide database. Hence, the AutoMSA program (AutoMSA v2.0) was again run.

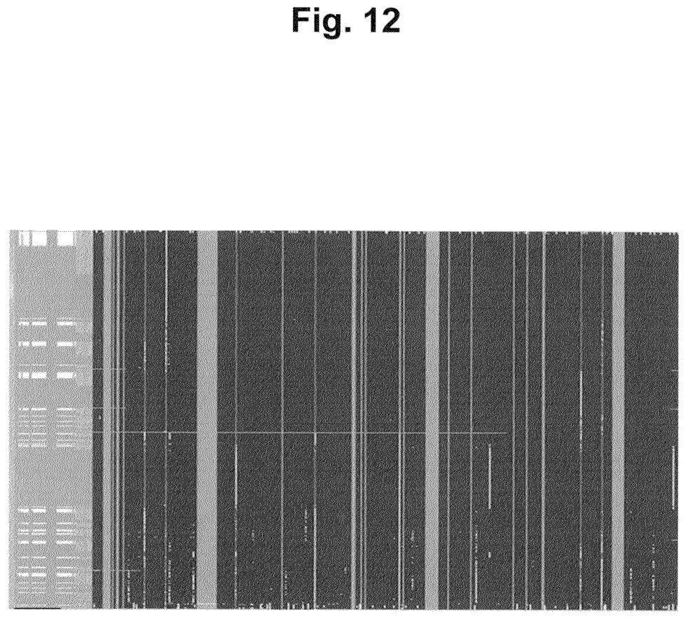

The user deleted, four times, group representative sequences 1, 3, and 7-24 with registration errors among the 25 group representative sequences selected in Comparative Example 1 and the target nucleic acid sequence data sets belonging to the same group as the group representative sequences, and then re-ran the AutoMSA program (AutoMSA v2.0) four times. As a result, the alignment results as shown in FIG. 12 were obtained.

In Comparative Example 2, five group representative sequences were selected through four times of sequence reviewing by the user, a total of 1,517 taxonomic names or taxonomic IDs were included in the finally retrieved target nucleic acid sequence data set, and the number of target nucleic acid sequence data finally retrieved was 13,798.

When comparing the results of Example 2 and the results of Comparative Example 2, the automated sequence retrieval method (AutoMSA v3.0) according to Example 2 selected five group representative sequences to provide an alignment file capable of designing an oligonucleotide, by running the program only once, and retrieved more target nucleic acid sequences than Comparative Example 2.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A computer-implemented method for providing a nucleic acid sequence data set for the design of an oligonucleotide used to detect a target nucleic acid molecule of an organism of interest, the method comprising:
(a) receiving a name of the target nucleic acid molecule and a name of the organism of interest and retrieving synonyms for the target nucleic acid molecule of the organism of interest;
(b) retrieving nucleic acid sequence data included in nucleic acid records, wherein each of the nucleic acid records is associated with the organism of interest and comprises a descriptor in which at least one of the name of the target nucleic acid molecule and the retrieved synonyms are described;
(c) sorting the retrieved nucleic acid data according to taxonomic name and/or taxonomic identification (ID) and selecting taxonomic representative sequences among nucleic acid sequence data having the same taxonomic name and/or taxonomic ID;
(d) grouping the selected taxonomic representative sequences according to homology and selecting a group representative sequence for each group; and
(e) retrieving nucleic acid sequence data having a homology of a predetermined value or more with the group representative sequence to provide the retrieved nucleic acid sequence data as a nucleic acid sequence data set for the design of an oligonucleotide.

2. The method according to claim 1, wherein in step (a), the name of the target nucleic acid molecule, a name of a protein encoded by the target nucleic acid molecule, and a name of an organism are received and the synonyms for the target nucleic acid molecule and the protein of the organism are retrieved.

3. The method according to claim 1, wherein the nucleic acid sequence data in step (b) include nucleic acid sequence data corresponding to a part or the entirety of the target nucleic acid molecule or variant nucleic acid sequence data for the target nucleic acid molecule.

4. The method according to claim 1, wherein the retrieving of nucleic acid sequence data in the step (b) is performed by the method comprising the following steps:
(b-1) retrieving identifiers of nucleic acid records, wherein each of the nucleic acid records is associated with the organism of interest and comprises a descriptor in which at least one of the name of the target nucleic acid molecule and the retrieved synonyms are described; and
(b-2) retrieving nucleic acid sequence data specified by the identifiers.

5. The method according to claim 1, wherein in step (b-1), the identifiers of the nucleic acid records are retrieved, wherein each of the nucleic acid records is associated with the organism of interest and comprises a descriptor in which at least one of the name of the target nucleic acid molecule, a name of a protein, and the retrieved synonyms are described.

6. The method according to claim 1, wherein in step (b-2), nucleic acid sequence data corresponding to the target nucleic acid molecule are selectively retrieved in the nucleic acid sequence data specified by the identifiers.

7. The method according to claim 1, wherein step (b-2) includes the following steps:
(b-2-1) retrieving nucleic acid records specified by the identifiers; and
(b-2-2) retrieving nucleic acid sequence data corresponding to the target nucleic acid molecule from the nucleic acid records.

8. The method according to claim 7, wherein in step (b-2-2), the nucleic acid sequence data corresponding to the target nucleic acid molecule and identification information of the nucleic acid sequence data are selectively retrieved from the nucleic acid records, wherein the selective retrieving of the nucleic acid sequence data and the identification information of the nucleic acid sequence data includes the following steps:
(b-2-2-1) determining, as a valid sub-record, a sub-record in which the synonym is recorded in a first specification that is predetermined, among one or more sub-records in each of the nucleic acid records;
(b-2-2-2) determining, as a valid sub-record, a sub-record in which the synonym is recorded in a second specification, when there is no valid sub-record determined by the first specification in the nucleic acid record;
(b-2-2-3) determining, as a valid sub-record, a sub-record in which the synonym is recorded in a third specification, when there is no valid sub-record determined by the second specification in the nucleic acid record; and
(b-2-2-4) retrieving nucleic acid sequence data corresponding to the determined valid sub-record and identification information thereof.

9. The method according to claim 1, wherein the method further comprises, between steps (b) and (c), the following steps:
(b-3) sorting the retrieved nucleic acid sequence data according to the biosample identification to select nucleic acid sequence data having the same biosample identification;
(b-4) sorting the selected nucleic acid sequence data so as to satisfy at least one of the following sort criteria;
(b-5) selecting a nucleic acid sequence of the highest-ranked nucleic acid sequence data among the sorted nucleic acid sequence data; and
(b-6) deleting nucleic acid sequence data except for the highest-ranked nucleic acid sequence data from the retrieved nucleic acid sequence data, wherein the sort criteria include the following:
(i) sorting the selected nucleic acid sequence data according to the assembly level, wherein as for the assembly level, complete genome, chromosome, scaffold, and config are ranked higher in that order; and
(ii) sorting the selected nucleic acid sequence data according to whether the selected nucleic acid sequence data are included in a reference sequence (RefSeq) database, wherein the ranking is higher when the nucleic acid sequence data are included in the RefSeq database than when not included.

10. The method according to claim 1, wherein the selecting of the taxonomic representative sequence in step (c) is performed by a method including the following steps:
(c-1) sorting the nucleic acid sequence data having the same taxonomic name and/or taxonomic ID so as to satisfy at least one of the following predetermined sort criteria; and
(c-2) selecting, as the taxonomic representative sequence, a nucleic acid sequence of the highest-ranked nucleic acid sequence data among the sorted nucleic acid sequence data, wherein the predetermined sort criteria include the following:
(i) sorting according to the assembly level of the nucleic acid sequence data, wherein as for the assembly level, complete genome, chromosome, scaffold, and config are ranked higher in that order;
(ii) sorting according to whether the nucleic acid sequence data are included in a reference sequence (RefSeq) database, wherein the ranking is higher when the nucleic acid sequence data are included in the RefSeq database than when not included;
(iii) sorting according to whether a name of a nucleic acid molecule described in a descriptor of a nucleic acid record containing the nucleic acid sequence data is identical to at least one of the received name of the target nucleic acid and the retrieved synonyms, wherein the ranking is higher when identical than when not identical;
(iv) sorting according to the length of the nucleic acid sequence data, wherein the longer the length, the higher the ranking;
(v) sorting according to whether there is description in a host of a descriptor of a nucleic acid record containing the nucleic acid sequence data, wherein the ranking is higher when a host of interest for the organism of interest is described in the host than when not described, and higher when an organism different from the organism of interest are not described in the host than when described;
(vi) sorting according to the registration date or revision date of a nucleic acid record containing the nucleic acid sequence data, wherein the more recent the registration date or revision date, the higher the ranking; and
(vii) sorting according to the alphabetical order in accession number of the nucleic acid sequence data, wherein the earlier the alphabetical order of the accession number, the higher the ranking.

11. The method according to claim 1, wherein the selecting of the group representative sequence in step (d) is performed by a method including the following steps:
(d-1) sorting the selected taxonomic representative sequences so as to satisfy at least one of the following predetermined sort criteria;
(d-2) selecting the highest-ranked taxonomic representative sequence among the sorted taxonomic representative sequences; and
(d-3) grouping taxonomic representative sequences having a homology of a predetermined value or more with the highest-ranked taxonomic representative sequence and selecting the highest-ranked taxonomic representative sequence as the group representative sequence in each group, wherein the sort criteria include the following:
(i) sorting according to the assembly level of the selected taxonomic representative sequence, wherein as for the assembly level, complete genome, chromosome, scaffold, and config are ranked higher in that order;
(ii) the number of nucleic acid sequence data having the same taxonomic name and/or taxonomic ID as the selected taxonomic representative sequence, wherein the larger the number, the higher the ranking;
(iii) sorting according to whether there is a description in a host of a descriptor of a nucleic acid record containing the selected taxonomic representative sequence, wherein the ranking is higher when a host of interest for the organism of interest is described in the host than when not described, and higher when an organism different from the host of interest is not described in the host than when described; and
(iv) sorting according to the alphabetic order of accession number in the selected taxonomic representative sequence, wherein the earlier the alphabetic order of the accession number, the higher the ranking.

12. The method according to claim 1, wherein the method further comprises step (f) of providing, as a target nucleic acid sequence data set for the target nucleic acid molecule, nucleic acid sequence data associated with the received organism of interest in the nucleic acid sequence data set provided in step (e).

13. The method according to claim 12, wherein the target nucleic acid sequence data set provided in step (f) has a homology of a predetermined value or more with at least one representative sequence of the group representative sequences and the taxonomic representative sequences for the target nucleic acid sequence data set.

14. The method according to claim 1, wherein the method further comprises step (g) of providing, as a non-target nucleic acid sequence data set for a non-target nucleic acid molecule, nucleic acid sequence data not associated with the received organism of interest from the nucleic acid sequence data set provided in step (e).

15. The method according to claim 14, wherein the non-target nucleic acid sequence data set provided in step (g) satisfies at least one of the following homology criteria:
(i) the non-target nucleic acid sequence data set needs to have a homology of a predetermined value or more with a partial sequence region of at least one representative sequence of the group representative sequence and the taxonomic representative sequence;
(ii) the non-target nucleic acid sequence data set needs to have a homology of a predetermined value or more with at least one representative sequence of the group representative sequence and the taxonomic representative sequence; and
(iii) the non-target nucleic acid sequence data set having homology criterion (i) needs to have homology criterion (ii).

16. The method according to claim 12, wherein the method further comprises the following steps:
(h) retrieving nucleic acid sequence data having a homology of a predetermined value or more with the group representative sequence to provide a nucleic acid sequence data set; and
(j) classifying, as design exclusion target nucleic acid sequence data, target nucleic acid sequence data of the group representative sequence and target nucleic acid sequence data belonging to the same group as the group representative sequence in the target nucleic acid sequence data set in step (f), when the taxonomic name and/or taxonomic ID of the organism associated with the group representative sequence satisfies one of the following predetermined criteria, wherein the predetermined criteria include the following:
(i) nucleic acid sequence data corresponding to the taxonomic name and/or taxonomic ID of the organism associated with the group representative sequence are absent and only nucleic acid sequence data corresponding to a taxonomic name and/or taxonomic ID of an organism different from the organism associated with the group representative sequence are present in the nucleic acid sequence data set provided in step (h);
(ii) the homology of nucleic acid sequence data corresponding to the taxonomic name and/or taxonomic ID of the organism associated with the group representative sequence is lower than the homology of nucleic acid sequence data corresponding to a taxonomic name and/or taxonomic ID of an organism different from the organism associated with the group representative sequence, in the nucleic acid sequence data set provided in step (h);
(iii) target nucleic acid sequence data corresponding to the taxonomic name and/or taxonomic ID of the organism associated with the group representative sequence are absent in the nucleic acid sequence data set provided in step (h), and the proportion of nucleic acid sequence data of an organism corresponding to a superclass taxonomic name and/or taxonomic ID of the organism associated with the group representative sequence or a subclass taxonomic name and/or taxonomic ID of the superclass is less than a predetermined value relative to the nucleic acid sequence data set provided in step (h); and
(iv) all of the nucleic acid sequence data set provided in step (h) is associated with a nucleic acid sequence data set of the organism associated with the group representative sequence, but a name of a target nucleic acid molecule described in a descriptor of each of nucleic acid records containing the nucleic acid sequence data set is absent or different from at least one of the name of the target nucleic acid molecule and the retrieved synonyms.

17. The method according to claim 1, wherein the method further comprises, after step (e), the following steps:
(e-1) sorting the provided nucleic acid sequence data set for the design according to the biosample identification to select nucleic acid sequence data having same biosample identification;
(e-2) sorting the selected nucleic acid sequence data so as to satisfy at least one of the following sort criteria;
(e-3) selecting a nucleic acid sequence of the highest-ranked nucleic acid sequence data among the sorted nucleic acid sequence data; and
(e-4) deleting nucleic acid sequence data except for the highest-ranked nucleic acid sequence data from the nucleic acid sequence data set for the design, wherein the sort criteria include the following:
(i) sorting nucleic acid sequences included in the provided nucleic acid sequence data set for the design according to the assembly level, wherein as for the assembly level, complete genome, chromosome, scaffold, and config are ranked higher in that order; and
(ii) sorting whether nucleic acid sequences included in the provided nucleic acid sequence data set for the design are included in a reference sequence (RefSeq) database, wherein the ranking is higher when the nucleic acid sequence data are included in the RefSeq database than when not included.

18. The method according to claim 14, wherein the method further comprises the following steps:
(k) retrieving nucleic acid sequence data having a homology of a predetermined value or more with the non-target nucleic acid sequence of the organism associated with the non-target nucleic acid sequence data set to provide a nucleic acid sequence data set; and
(l) classifying, as design exclusion non-target nucleic acid sequence data, non-target nucleic acid sequence data of the organism in the non-target nucleic acid sequence data set in step (k), when the taxonomic name and/or taxonomic ID of the organism associated with the non-target nucleic acid sequence satisfies one criterion of the following predetermined criteria, wherein the predetermined criteria include the following:
(i) nucleic acid sequence data corresponding to the taxonomic name and/or taxonomic ID of the organism associated with the non-target nucleic acid sequence are absent and only nucleic acid sequence data corresponding to a taxonomic name and/or taxonomic ID of an organism different from the organism are present in the nucleic acid sequence data set provided in step (k);
(ii) the homology of the non-target nucleic acid sequence data corresponding to the taxonomic name and/or taxonomic ID of the organism associated with the non-target nucleic acid sequence is lower than the homology of nucleic acid data corresponding to a taxonomic name and/or taxonomic ID of an organism different from the organism in the nucleic acid sequence data set provided in step (k); and
(iii) non-target nucleic acid sequence data corresponding to the taxonomic name and/or taxonomic ID of the organism associated with the non-target nucleic acid sequence are absent in the nucleic acid sequence data set provided in step (k), and the proportion of nucleic acid sequence data of an organism corresponding to a superclass taxonomic name and/or taxonomic ID of the organism or a subclass taxonomic name and/or taxonomic ID of the superclass is less than a predetermined value relative to the nucleic acid sequence data set provided in step (k).

19. A computer readable storage medium containing instructions to configure a processor to perform a method for providing a nucleic acid sequence data set for the design of an oligonucleotide used to detect a target nucleic acid molecule of an organism of interest, the method comprising:
(a) receiving a name of the target nucleic acid molecule and a name of the organism of interest and retrieving synonyms for the target nucleic acid molecule of the organism of interest;
(b) retrieving nucleic acid sequence data included in nucleic acid records, wherein each of the nucleic acid records is associated with the organism of interest and comprises a descriptor in which at least one of the name of the target nucleic acid molecule and the retrieved synonyms are described;
(c) sorting the retrieved nucleic acid data according to taxonomic name and/or taxonomic identification (ID) and selecting taxonomic representative sequences among nucleic acid sequence data having the same taxonomic name and/or taxonomic ID;
(d) grouping the selected taxonomic representative sequences according to homology and selecting a group representative sequence for each group; and
(e) retrieving nucleic acid sequence data having a homology of a predetermined value or more with the group representative sequence to provide the retrieved nucleic acid sequence data as a nucleic acid sequence data set for the design of an oligonucleotide.
